(19) United States
(12) Patent Application Publication
(10) Pub. No.: US 2023/0218666 A1
(43) Pub. Date: Jul. 13, 2023

(54) ANTIMICROBIAL COMBINATIONS

(71) Applicant: MARVELBIOTICS LIMITED, Nether Alderley Cheshire (GB)

(72) Inventors: Ronald SHANNON, Nether Alderley Cheshire (GB); Natalia KIRSTEN, Nether Alderley Cheshire (GB)

(21) Appl. No.: 18/002,203

(22) PCT Filed: Jun. 18, 2021
(86) PCT No.: PCT/GB2021/051552

§ 371 Date: Dec. 16, 2022

(30) Foreign Application Priority Data
Jun. 19, 2020 (GB) 2009421.5
Mar. 3, 2021 (GB) 2102993.9

(51) Int. Cl. A61K 33/34; A61K 33/30; A61K 36/064; A61K 35/745; A61K 35/747; A61K 33/00; A61P 31/04; A61P 31/10

(52) U.S. Cl. CPC A61K 33/34; A61K 33/30; A61K 36/064; A61K 35/745; A61K 35/747; A61K 33/00; A61P 31/04; A61P 31/10

(57) ABSTRACT

Aspects of the present invention relate inter alia to compositions which may have utility as anti-microbials. Such antimicrobial compositions may be for use in promoting wound healing and/or the treatment of microbial infections. In certain embodiments, the invention relates to an antimicrobial composition and/or combinations comprising copper gluconate and zinc gluconate which may be for use in treating and/or preventing infections and/or promoting wound healing. Certain aspects of the present invention further elate to an antimicrobial composition and/or device comprising copper gluconate, zinc gluconate and Lactobacillus plantarum for use in treating infections and/or promoting wound healing. Also encompassed by the present invention are methods of treating microbial and/or fungal infections in a subject, for example infections of the genital tract. Aspects of the present invention include a composition comprising a zinc salt and a copper salt, wherein the zinc salt is zinc gluconate and the copper salt is copper gluconate.

### FIELD OF THE INVENTION

Aspects of the present invention relate inter alia to compositions which may have utility as anti-microbials. Such antimicrobial compositions may be for use in promoting wound healing and/or the treatment of microbial infections. In certain embodiments, the invention relates to an antimicrobial composition and/or combinations comprising copper gluconate and zinc gluconate which may be for use in treating and/or preventing infections and/or promoting wound healing. Certain aspects of the present invention further relate to an antimicrobial composition and/or device comprising copper gluconate, zinc gluconate and Lactobacillus plantarum for use in treating infections and/or promoting wound healing. Also encompassed by the present invention are methods of treating microbial and/or fungal infections in a subject, for example infections of the genital tract.

### BACKGROUND TO THE INVENTION

Infectious diseases are one of the leading causes of deaths worldwide. Pathologically these conditions involve infection of the body by disease-causing microbial pathogens, which are highly adapted microorganisms—including bacteria, virus, parasite, and fungi—that have the capacity to cause disease.

Such pathogenic micro-organisms can adhere to, invade, and cause damage to host cells and tissues. During the prolonged course of their evolution, disease-causing pathogenic microbes have developed a variety of virulence mechanisms to cause disease in the host, including the secretion and presentation of toxic agents. Microbial infections and the conditions they cause vary immensely, from mild disorders to life-threatening illnesses. For example, common bacterial infections include pneumonia, ear infections, diarrhoea, genital tract infections, urinary tract infections, and skin disorders.

The host organism naturally possesses barriers to prevent infection by pathogens e.g skin, mucous membranes, tears, earwax, mucus, and stomach acid. Further the host's immune system comprises an innate immune response to immediately combat the invasion of pathogenic micro-organisms. Therefore, successful microbial infection reflects not only the persistence within a host organism required for disease, but also the ability of the pathogen to overcome and evade the host's barriers and immune response.

At present, the standard treatment prescribed by clinicians for bacterial infections is a course of antibiotics. Antibiotics are antimicrobial agents used to fight a broad range of bacterial infections. These drugs kill and/or inhibit the growth of bacteria by several methods including: targeting the bacterial cell's wall or outer coating, interfering with bacteria reproduction, or blocking protein production within the bacterial cell. The value of antibiotics to public health cannot be understated, prior to their discovery and medical use, around 30 percent of all deaths were caused by bacterial infections.

Unfortunately, the use of antibiotics naturally selects for bacteria that have evolved or acquired antibiotic resistance. Antibiotic resistance leads to higher medical costs, prolonged hospital stays, and increased mortality. The capacity of the bacterial cell to survive the antibiotic dose may result from: limiting uptake of the drug, modification of the drug target, inactivation of the drug, and active efflux of the drug. Not only is this resistance typically inheritable, but the capability of bacteria to perform horizontal gene transmission renders the spread of antibiotic-resistant phenotypes a huge challenge to the long-term use of antibiotics to effectively treat microbial infections.

Although antibiotic resistance occurs naturally, the issue has been intensified by the over-prescription of antibiotics by clinicians, which together with the premature ending of treatment regimens by patients, has fast-tracked the rise in antimicrobial resistance. To the point where at present, a growing number of infections are becoming continually harder to treat, as the antibiotics typically used have become less and less effective, with certain bacterial strains and species now having no effective treatments of any kind. As such antimicrobial resistance—and the associated risk of a post-antibiotic era—is recognized as one of the biggest threats to global health, food security, and development today.

Consequently, there is a continuing need in the art for effective methods of treating microbial infections and/or improving the current methods of treating these infections. Particularly relating to the growing prevalence of antimicrobial-resistant bacterial strains, which presents a formidable threat to public health.

It is an aim of certain embodiments of the present invention to at least partially mitigate the problems associated with the prior art.

It is an aim of certain embodiments of the present invention to provide an antimicrobial composition and/or device.

It is an aim of certain embodiments of the present invention to provide an antimicrobial composition for the treatment and/or prevention of infections.

### SUMMARY OF CERTAIN EMBODIMENTS OF THE INVENTION

There remains a clear need to identify novel combinations of antimicrobial agents, which can be used, for example, to eliminate and/or displace pathogenic microbes from the skin and/or mucosa—yet tolerate the presence of commensal micro-organisms, and so be able to protect and/or restore the microbiome. The development of such antimicrobial interventions would be of great value in the treatment and/or prevention of infection, and so would be beneficial, for example, in wound healing, particularly for chronic or non-healing wounds. In fact, these new compositions and devices may also comprise a commensal micro-organism;

the presence of which may further enhance the compositions use in treating and/or preventing infections.

In a broadest aspect, the present invention provides anti-microbial compositions and uses thereof. The compositions of certain embodiments of the present invention may have utility in the treatment and/or prevention of infections by one or more of the following bacteria:
a) Acinetobacter baumannii;
b) Campylobacter coli;
c) Haemophilus influenzae;
d) Helicobacter aeruginosa;
e) Pseudomonas aeruginosa;
f) Salmonella enteritidis;
g) Enterococcus faecium;
h) Neisseria gonorrhoeae;
i) Staphylococcus aureus; and
j) Streptococcus pneumoniae.

The compositions of certain embodiments of the present invention may have utility in the treatment and/or prevention of infections by one or more of the following micro-organisms:
a) Acinetobacter baumannii;
b) Campylobacter coli;
c) Haemophilus influenzae;
d) Helicobacter aeruginosa;
e) Pseudomonas aeruginosa;
f) Salmonella enteritidis;
g) Enterococcus faecium;
h) Neisseria gonorrhoeae;
i) Stomatococcus mucilaginous;
j) Staphylococcus aureus;
k) Staphylococcus hominis;
I) Streptococcus pneumoniae;
m) Streptococcus mutans;
n) Cutibacterium;
o) Anaerococcus;
p) Peptoniphilus;
q) Candida albicans;
r) Malassezia restricta;
s) Malassezia globosa;
t) Propionibacterium;
u) Gardnerella vaginalis;
v) Escherichia coli;
w) Prevotella;
x) Peptostreptococcus;
y) Bacteroides; and
z) Corynebacterium.

In some embodiments, the composition may be used to treat a fungal infection, such as but not limited to a Candida infection e.g. Candida albicans and/or Candida auris.

In a further aspect, the present invention provides a combination of components which may have utility in the treatment and/or prevention of infections by one or more of the following bacteria:
a) Acinetobacter baumannii;
b) Campylobacter coli;
c) Haemophilus influenzae;
d) Helicobacter aeruginosa;
e) Pseudomonas aeruginosa;
f) Salmonella enteritidis;
g) Enterococcus faecium;
h) Neisseria gonorrhoeae;
i) Staphylococcus aureus; and
j) Streptococcus pneumoniae.

The antimicrobial compositions and combinations of certain embodiments may have utility in the treatment and/or prevention of infections. Aptly, aspects of the invention provide combinations and compositions for modulating the microbial load present at a site of infection. Aptly, aspects of the present invention provide methods and compositions for use in the treatment and/or prevention of genital tract infections. In certain embodiments, the methods and compositions are for use in preventing and/or treating yeast infections e.g. Candida infections.

The antimicrobial compositions and combinations of certain embodiments may have utility in the promotion of wound healing. Aptly, aspects of the present invention provide compositions for modulating the microbial load present in the wound site.

In an aspect of the present invention there is provided an antimicrobial composition and/or device comprising copper gluconate and zinc gluconate.

In an aspect of the present invention there is provided a combination comprising copper gluconate and zinc gluconate.

The composition and/or the combination can be used to promote wound healing. In further embodiments the wound may be a chronic or non-healing wound. Aptly, the wound can be caused by a trauma, ulcer and/or a burn.

In certain embodiments, the present invention provides an antimicrobial composition comprising copper gluconate, zinc gluconate and Lactobacillus plantarum. The composition may be used to treat and/or prevent infections—and so promote wound healing—in a topical formulation and/or device.

In certain embodiments, the present invention provides a combination comprising copper gluconate, zinc gluconate and Lactobacillus plantarum. The combination can be used to treat and/or prevent infections—and so promote wound healing—in a topical formulation and/or device.

In some embodiments the combination comprises two or more products, wherein one or more products comprise copper gluconate and one or more products comprise zinc gluconate and one or more products comprise Lactobacillus plantarum. In certain embodiments, one product may comprise one or more of copper gluconate, zinc gluconate and Lactobacillus plantarum and a further product may comprise copper gluconate, zinc gluconate and Lactobacillus plantarum. In certain embodiments, the combination comprises at least one product which is a first product and at least one product which is a further product, wherein the first and further products are different forms. In certain embodiments, the first and further products may be configured for use at different times. In certain embodiments, the combination may comprise a cream, a gel and/or a lotion comprising Lactobacillus plantarum and an aerosol foam or spray comprising copper gluconate and zinc gluconate. In certain embodiments, the combination may comprise a cream, a gel and/or a lotion comprising copper gluconate and zinc gluconate and an aerosol foam or spray comprising Lactobacillus plantarum. Aptly, the first product e.g. the cream and the second product e.g. the spray and/or foam are for separate administration to subject's infection site, wound, or area of treatment. In such embodiments the first and further products may be applied to the subject consecutively e.g. within about five minutes, ten minutes, fifteen minutes or twenty minutes between application of the first product and the further product. Alternatively, in certain embodiments there may be a time period between application of the combination's separate forms. In some embodiments, the time period between application of the separate forms e.g. the first product and the further product is less than 1 hour, less than 2 hours, less than 3 hours, less than 4 hours, less than 5 hours, less than 6 hours, less than 7 hours, less than 8 hours, less than 9 hours, less than 10 hours, less than 11 hours, less than 12 hours, less than 24 hours, less than 36 hours, less than 48 hours or less than 72 hours.

In certain embodiments, the combination is for use in the treatment of a skin condition e.g. psoriasis and/or eczema. In certain embodiments, the combination is for use in the treatment of a wound. The wound may be for example a laceration, a surgical incision, an ulcer or a burn.

In certain embodiments, the composition and/or combination is for the prevention and/or treatment of a vaginal disorder such as bacterial vaginosis.

In an aspect of the present invention there is provided a composition comprising (a) zinc salt and (b) copper salt, wherein the zinc salt is zinc gluconate and the copper salt is copper gluconate.

In a further aspect of the present invention there is provided a combination comprising (a) zinc salt and (b) copper salt, wherein the zinc salt is zinc gluconate and the copper salt is copper gluconate.

In certain embodiments, the composition and/or combination comprises zinc gluconate in a concentration of at least 0.05% w/v e.g. 0.055, 0.06, 0.065, 0.07, 0.075, 0.08, 0.085, 0.09, 0.095%.

In certain embodiments, the composition and/or combination comprises zinc gluconate in a concentration of approx. 0.1% w/v.

In certain embodiments, the composition and/or combination comprises zinc gluconate in a concentration of greater than 0.1% w/v, e.g. 0.125, 0.13, 0.014, 0.015, 0.016, 0.017, 0.018, 0.019%, 0.02% w/v or greater.

In certain embodiments, the composition and/or combination comprises copper gluconate in a concentration of at least 0.1% w/v e.g. 0.125, 0.13, 0.014, 0.015, 0.016, 0.017, 0.018, 0.019% w/v or greater.

In certain embodiments, the composition and/or combination comprises copper gluconate in a concentration of about 0.2% w/v.

In certain embodiments, the composition and/or combination comprises copper gluconate in a concentration of greater than 0.2% e.g. 0.21, 0.22, 0.23, 0.24, 0.25, 0.26, 0.27, 0.28, 0.29 or 0.30% w/v or greater.

In certain embodiments, the composition further comprises one or more further copper components, for example, copper sulfate, a copper-complex e.g. a copper chelates e.g. copper histidine.

In certain embodiments, the composition further comprises one or more further zinc components e.g. zinc sulfate and/or one or more zinc complexes e.g. zinc histidine.

In certain embodiments, the composition further comprises one or more titanium compounds e.g. titanium dioxide.

In certain embodiments, the composition further comprises one or more further pro-biotic bacteria and/or yeast.

In certain embodiments, the combination further comprises one or more further copper components, for example, copper sulfate, a copper-complex e.g. a copper chelates e.g. copper histidine.

In certain embodiments, the combination further comprises one or more further zinc components e.g. zinc sulfate and/or one or more zinc complexes e.g. zinc histidine.

In certain embodiments, the combination further comprises one or more titanium compounds e.g. titanium dioxide.

In certain embodiments, the combination further comprises one or more further pro-biotic bacteria and/or yeast.

In certain embodiments, the probiotic bacteria is selected from Lactobacillus species, bifidobacterium species, e.g. Lactobacillus rhamnosus, Lactobacillus casei, Lactobacillus acidophilus and Lactobacillus lactus.

In certain embodiments, the probiotic yeast is Saccharomyces boulardii.

In certain embodiments, the composition is a suspension.

In certain embodiments, the composition further comprises Lactobacillus plantarum.

In certain embodiments, the composition comprises Lactobacillus plantarum at a concentration of about 1×10⁸±5×107 CFUmL−1

Aptly, the Lactobacillus plantarum is a sterilized Lactobacillus plantarum.

In certain embodiments, the probiotic bacteria are in the form of viable microorganisms.

In certain embodiments, the probiotic bacteria are in the form of lysates.

In certain embodiments, the probiotic bacteria are in the form of tyndallizates.

In a further aspect of the present invention, there is provided a composition of the first aspect for use as an anti-microbial agent.

In certain embodiments, the composition and/or combination is for use in the treatment of an infection in a subject of one or more of the following bacterial organisms:
a) Acinetobacter baumannii;
b) Campylobacter coli;
c) Haemophilus influenzae;
d) Helicobacter aeruginosa;
e) Pseudomonas aeruginosa;
f) Salmonella enteritidis;
g) Enterococcus faecium;
h) Neisseria gonorrhoeae;
i) Staphylococcus aureus; and
j) Streptococcus pneumoniae.

In certain embodiments, the composition and/or combination is for use in the treatment of an infection and/or colonization in a subject of one or more of the following micro-organisms:
a) Acinetobacter baumannii;
b) Campylobacter coli;
c) Haemophilus influenzae;
d) Helicobacter aeruginosa;
e) Pseudomonas aeruginosa;
f) Salmonella enteritidis;
g) Enterococcus faecium;
h) Neisseria gonorrhoeae;
i) Stomatococcus mucilaginous;
j) Staphylococcus aureus;
k) Staphylococcus hominis;
I) Streptococcus pneumoniae;
m) Streptococcus mutans;
n) Cutibacterium;
o) Anaerococcus;
p) Peptoniphilus;
q) Candida albicans;
r) Malassezia restricta;
s) Malassezia globosa;
t) Propionibacterium;
u) Gardnerella vaginalis;
v) Escherichia coli;
w) Prevotella;
x) Peptostreptococcus;
y) Bacteroides; and
z) Corynebacterium.

In certain embodiments, the composition and/or combination is for use in the treatment of an Acinetobacter baumannii infection in a subject.

In certain embodiments, the composition and/or combination is for use in the treatment of a Campylobacter coli infection in a subject.

In certain embodiments, the composition and/or combination is for use in the treatment of a Haemophilus influenzae infection in a subject.

In certain embodiments, the composition and/or combination is for use in the treatment of a Helicobacter aeruginosa infection in a subject.

In certain embodiments, the composition and/or combination is for use in the treatment of a Pseudomonas aeruginosa infection in a subject.

In certain embodiments, the composition and/or combination is for use in the treatment of a Salmonella enteritidis infection in a subject.

In certain embodiments, the composition and/or combination is for use in the treatment of a Enterococcus faecium infection in a subject.

In certain embodiments, the composition and/or combination is for use in the treatment of a Neisseria gonorrhoeae infection in a subject.

In certain embodiments, the composition and/or combination is for use in the treatment of a Staphylococcus aureus infection in a subject.

In certain embodiments, the composition and/or combination is for use in the treatment of a Streptococcus pneumoniae infection in a subject.

In certain embodiments, the composition and/or combination is for use in the treatment of a disorder characterised by colonization by C. albicans, C. auris and/or A. brasiliensis.

In certain embodiments, the composition and/or combination is for use in the treatment of a disorder characterized by colonization by S. aureus and P. aeruginosa.

In certain embodiments, the composition and/or combination is for use in the treatment of an infection by and/or a disorder characterised by colonization of Stomatococcus mucilaginous in a subject.

In certain embodiments, the composition and/or combination is for use in the treatment of an infection by and/or a disorder characterised by colonization of Staphylococcus hominis in a subject.

In certain embodiments, the composition and/or combination is for use in the treatment of an infection by and/or a disorder characterised by colonization of Streptococcus mutans in a subject.

In certain embodiments, the composition and/or combination is for use in the treatment of an infection by and/or a disorder characterised by colonization of Cutibacterium in a subject.

In certain embodiments, the composition and/or combination is for use in the treatment of an infection by and/or a disorder characterised by colonization of Anaerococcus in a subject.

In certain embodiments, the composition and/or combination is for use in the treatment of an infection by and/or a disorder characterised by colonization of Peptoniphilus in a subject.

In certain embodiments, the composition and/or combination is for use in the treatment of an infection by and/or a disorder characterised by colonization of Candida albicans in a subject.

In certain embodiments, the composition and/or combination is for use in the treatment of an infection by and/or a disorder characterised by colonization of Malassezia restricta in a subject.

In certain embodiments, the composition and/or combination is for use in the treatment of an infection by and/or a disorder characterised by colonization of Malassezia globosa in a subject.

In certain embodiments, the composition and/or combination is for use in the treatment of an infection by and/or a disorder characterised by colonization of Propionibacterium in a subject.

In certain embodiments, the composition and/or combination is for use in the treatment of an infection by and/or a disorder characterised by colonization of Gardnerella vaginalis in a subject.

In certain embodiments, the composition and/or combination is for use in the treatment of an infection by and/or a disorder characterised by colonization of Escherichia coli in a subject.

In certain embodiments, the composition and/or combination is for use in the treatment of an infection by, and/or a disorder characterised by colonization of Prevotella in a subject.

In certain embodiments, the composition and/or combination is for use in the treatment of an infection by and/or a disorder characterised by colonization of Peptostreptococcus in a subject.

In certain embodiments, the composition and/or combination is for use in the treatment of an infection by and/or a disorder characterised by colonization of Bacteroides in a subject.

In certain embodiments, the composition and/or combination is for use in the treatment of an infection by and/or a disorder characterised by colonization of Corynebacterium in a subject.

In certain embodiments, the composition and/or combination is for use to treat a wound which is colonized by one or more of the following micro-organisms:
a) Acinetobacter baumannii;
b) Campylobacter coli;
c) Haemophilus influenzae;
d) Helicobacter aeruginosa;
e) Pseudomonas aeruginosa;
f) Salmonella enteritidis;
g) Enterococcus faecium;
h) Neisseria gonorrhoeae;
i) Staphylococcus aureus; and
j) Streptococcus pneumoniae.

In certain embodiments, the composition and/or combination is for use to treat a disorder characterised by colonization by one or more of the following micro-organisms:
a) Acinetobacter baumannii;
b) Campylobacter coli;
c) Haemophilus influenzae;
d) Helicobacter aeruginosa;
e) Pseudomonas aeruginosa;
f) Salmonella enteritidis;
g) Enterococcus faecium;
h) Neisseria gonorrhoeae;
i) Stomatococcus mucilaginous;
j) Staphylococcus aureus;
k) Staphylococcus hominis;
I) Streptococcus pneumoniae;
m) Streptococcus mutans;
n) Cutibacterium;
o) Anaerococcus;
p) Peptoniphilus;
q) Candida albicans;
r) Malassezia restricta;
s) Malassezia globosa;
t) Propionibacterium;
u) Gardnerella vaginalis;
v) Escherichia coli;
w) Prevotella;
x) Peptostreptococcus;
y) Bacteroides; and
z) Corynebacterium.

In certain embodiments, the wound is a skin wound selected from the group consisting of a laceration, a penetrating wound, a venous stasis ulcer, a pressure ulcer, a surgical wound and an open full-thickness skin puncture wound.

In certain embodiments, the composition and/or combination is for use in the treatment of a genital tract disorder. Aptly, the genital tract disorder is a genital tract infection. In certain embodiments, the composition and/or combination is for use in the prevention of a genital tract disorder. In certain embodiments, the composition and/or combination is for use in the prevention of a genital tract disorder such as but not limited to bacterial vaginosis.

In certain embodiments, the composition and/or combination is for use in the treatment of a fungal infection in a subject, wherein optionally the fungal infection is a Candida infection.

In certain embodiments, the composition and/or combination is for use in the treatment of a Candida albicans and/or a Candida auris infection in a subject. Aptly, the composition is for use to treat an oral Candida albicans and/or a Candida auris infection, and/or a vaginal Candida albicans and/or a Candida auris infection.

In certain embodiments, the composition and/or combination is for topical, oral and/or parenteral administration.

In certain embodiments, the composition and/or combination is for topical administration.

In certain embodiments, the composition and/or combination is for use to prevent or reduce the formation of a biofilm.

In a further aspect of the present invention, there is provided a method of treating and/or preventing a microbial infection in a subject, the method comprising:
a) administering a composition or combination as described herein to a subject in need thereof.

In certain embodiments, the microbial infection is selected from the group consisting of:
a) Acinetobacter baumannii;
b) Campylobacter coli;
c) Haemophilus influenzae;
d) Helicobacter aeruginosa;
e) Pseudomonas aeruginosa;
f) Salmonella enteritidis;
g) Enterococcus faecium;
h) Neisseria gonorrhoeae;
i) Staphylococcus aureus; and
j) Streptococcus pneumoniae.

In certain embodiments, the microbial infection is selected from the group consisting of:
a) Acinetobacter baumannii;
b) Campylobacter coli;
c) Haemophilus influenzae;
d) Helicobacter aeruginosa;
e) Pseudomonas aeruginosa;
f) Salmonella enteritidis;
g) Enterococcus faecium;
h) Neisseria gonorrhoeae;
i) Stomatococcus mucilaginous;
j) Staphylococcus aureus;
k) Staphylococcus hominis;
I) Streptococcus pneumoniae;
m) Streptococcus mutans;
n) Cutibacterium;
o) Anaerococcus;
p) Peptoniphilus;
q) Candida albicans;
r) Malassezia restricta;
s) Malassezia globosa;
t) Propionibacterium;
u) Gardnerella vaginalis;
v) Escherichia coli;
w) Prevotella;
x) Peptostreptococcus;
y) Bacteroides; and
z) Corynebacterium.

In a further aspect of the present invention, there is provided a method of treating a wound, lesion and/or burn suspected of being infected by a micro-organism and/or preventing infection of a wound, lesion and/or burn of a subject by a micro-organism, the method comprising:
a) applying a composition or combination as described herein to a site of a wound, a lesion and/or a burn of a subject in need thereof.

In certain embodiments, the composition and/or combination is for the treatment of a wound which is a skin wound selected from the group consisting of a laceration, a penetrating wound, a venous stasis ulcer, a pressure ulcer, a surgical wound and an open full-thickness skin puncture wound.

In certain embodiments, there is provided a method of treating and/or preventing a genital tract infection, the method comprising:
a) administering a composition or a combination as described herein to a site in the genital tract of a subject in need thereof.

In certain embodiments, the composition and/or combination as described herein is for the treatment and/or prevention of one or more of the following:
(a) body odour;
(b) dandruff;
(c) dental caries;
(d) genital infections; and
(e) halitosis.

In certain embodiments, the composition and/or combination as described herein is a chewable product.

In certain embodiments, the method comprises applying the composition or the combination to a site infected with or suspected of being infected with a micro-organism.

In certain embodiments, the method comprises applying the composition in the form of a cream, a gel and/or a lotion.

In certain embodiments, the method comprises repeating the administration of the composition to the site.

### BRIEF DESCRIPTION OF DRAWINGS

Certain embodiments of the present invention will now be described hereinafter, by way of example only, with reference to the accompanying drawings in which:

FIG. 1 is a graph showing the average quantities of viable Acinetobacter baumannii recovered per sample from test agents compared to the negative control at 0 hours. Formulation 1-0.2% Copper gluconate and 0.1% zinc gluconate with Lactobacillus plantarum. Formulation 2-0.2% Copper gluconate and 0.1% zinc gluconate without Lactobacillus plantarum. Error bars represent standard deviation. Red line (linear line) represents limit of detection (1 Log).

FIG. 2 is a graph showing the average quantities of viable Campylobacter coli recovered per sample from test agents compared to the negative control at 0 hours. Formulation 1—0.2% Copper gluconate and 0.1% zinc gluconate with Lactobacillus plantarum. Formulation 2-0.2% Copper gluconate and 0.1% zinc gluconate without Lactobacillus plantarum. Error bars represent standard deviation. Red line (linear line) represents limit of detection (1 Log).

FIG. 3 is a graph showing the average quantities of viable Enterobacter cloacae recovered per sample from test agents compared to the negative control at 0 hours. Formulation 1—0.2% Copper gluconate and 0.1% zinc gluconate with Lactobacillus plantarum. Formulation 2-0.2% Copper gluconate and 0.1% zinc gluconate without Lactobacillus plantarum. Error bars represent standard deviation. Red line (linear line) represents limit of detection (1 Log).

FIG. 4 is a graph showing the average quantities of viable Haemophilus influenzae per sample recovered from test agents compared to the negative control at 0 hours. Formulation 1-0.2% Copper gluconate and 0.1% zinc gluconate with Lactobacillus plantarum. Formulation 2-0.2% Copper gluconate and 0.1% zinc gluconate without Lactobacillus plantarum. Error bars represent standard deviation. Red line (linear line) represents limit of detection (1 Log).

FIG. 5 is a graph showing the average quantities of viable Helicobacter pylori per sample recovered from test agents compared to the negative control at 0 hours. Formulation 1—0.2% Copper gluconate and 0.1% zinc gluconate with Lactobacillus plantarum. Formulation 2-0.2% Copper gluconate and 0.1% zinc gluconate without Lactobacillus plantarum. Error bars represent standard deviation. Red line (linear line) represents limit of detection (1 Log).

FIG. 6 is a graph showing the (phase 3) average quantities of viable Pseudomonas aeruginosa recovered per sample from test agents compared to the negative control. Formulation 1-0.2% Copper gluconate and 0.1% zinc gluconate with Lactobacillus plantarum. Formulation 2-0.2% Copper gluconate and 0.1% zinc gluconate without Lactobacillus plantarum. Error bars represent standard deviation. Red line (linear line) represents limit of detection (1 Log).

FIG. 7 is a graph showing the (phase 5) average quantities of viable Pseudomonas aeruginosa per sample recovered from test agents compared to the negative control at 0 hours. Formulation 1-0.2% Copper gluconate and 0.1% zinc gluconate with Lactobacillus plantarum. Formulation 2-0.2% Copper gluconate and 0.1% zinc gluconate without Lactobacillus plantarum. Error bars represent standard deviation. Red line (linear line) represents limit of detection (1 Log).

FIG. 8 is a graph showing the average quantities of viable Salmonella enteritidis per sample recovered from test agents compared to the negative control at 0 hours. Formulation 1-0.2% Copper gluconate and 0.1% zinc gluconate with Lactobacillus plantarum. Formulation 2-0.2% Copper gluconate and 0.1% zinc gluconate without Lactobacillus plantarum. Error bars represent standard deviation. Red line (linear line) represents limit of detection (1 Log).

FIG. 9 is a graph showing the average quantities of viable Enterococcus faecium recovered per sample from test agents compared to the negative control at 0 hours. Formulation 1-0.2% Copper gluconate and 0.1% zinc gluconate with Lactobacillus plantarum. Formulation 2-0.2% Copper gluconate and 0.1% zinc gluconate without Lactobacillus plantarum. Error bars represent standard deviation. Red line (linear line) represents limit of detection (1 Log).

FIG. 10 is a graph showing the average quantities of viable Neisseria gonorrhoeae per sample recovered from test agents compared to the negative control at 0 hours. Formulation 1-0.2% Copper gluconate and 0.1% zinc gluconate with Lactobacillus plantarum. Formulation 2-0.2% Copper gluconate and 0.1% zinc gluconate without Lactobacillus plantarum. Error bars represent standard deviation. Red line (linear line) represents limit of detection (1 Log).

FIG. 11 is a graph showing the (phase 3) average quantities of viable Staphylococcus aureus recovered per sample from test agents compared to the negative control. Formulation 1-0.2% Copper gluconate and 0.1% zinc gluconate with Lactobacillus plantarum. Formulation 2-0.2% Copper gluconate and 0.1% zinc gluconate without Lactobacillus plantarum. Error bars represent standard deviation. Red line (linear line) represents limit of detection (1 Log).

FIG. 12 is a graph showing the (phase 5) average quantities of viable Staphylococcus aureus per sample recovered from test agents compared to the negative control at 0 hours. Formulation 1-0.2% Copper gluconate and 0.1% zinc gluconate with Lactobacillus plantarum. Formulation 2-0.2% Copper gluconate and 0.1% zinc gluconate without Lactobacillus plantarum. Error bars represent standard deviation. Red line (linear line) represents limit of detection (1 Log).

FIG. 13 is a graph showing the average quantities of viable Streptococcus pneumoniae per sample recovered from test agents compared to the negative control at 0 hours. Formulation 1-0.2% Copper gluconate and 0.1% zinc gluconate with Lactobacillus plantarum. Formulation 2-0.2% Copper gluconate and 0.1% zinc gluconate without Lactobacillus plantarum. Error bars represent standard deviation. Red line (linear line) represents limit of detection (1 Log).

FIG. 14 is a graph showing the average quantities of viable Candida albicans recovered per sample from test agents compared to the negative control. Formulation 1-0.2% Copper gluconate and 0.1% zinc gluconate with Lactobacillus plantarum. Formulation 2-0.2% Copper gluconate and 0.1% zinc gluconate without Lactobacillus plantarum. Error bars represent standard deviation. Red line (linear line) represents limit of detection (1 Log).

FIG. 15 is a graph showing the average quantities of viable Candida auris recovered per sample from test agents compared to the negative control. Formulation 1-0.2% Copper gluconate and 0.1% zinc gluconate with Lactobacillus plantarum. Formulation 2-0.2% Copper gluconate and 0.1% zinc gluconate without Lactobacillus plantarum. Error bars represent standard deviation. Red line (linear line) represents limit of detection (1 Log).

FIG. 16 is a graph showing the average quantities of viable Aspergillus brasiliensis per sample recovered from test agents compared to the negative control. Formulation 1-0.2% Copper gluconate and 0.1% zinc gluconate with Lactobacillus plantarum. Formulation 2—0.2% Copper gluconate and 0.1% zinc gluconate without Lactobacillus plantarum. Error bars represent standard deviation. Red line (linear line) represents limit of detection (1 Log).

FIG. 17 is a graph showing the average quantities of Staphylococcus aureus recovered from a pre-formed biofilm (24 hour), following 24 hours treatment with Formulation 1-0.2% copper gluconate and 0.1% zinc gluconate with Lactobacillus plantarum and Formulation 2—0.2% copper gluconate and 0.1% zinc gluconate, and control solutions. Red line (linear line) represents the limit of detection (1 Log). Error bars represent standard deviations.

FIG. 18 is a graph showing the average quantities of Pseudomonas aeruginosa recovered from a pre-formed biofilm (24 hour), following 24 hours treatment with Formulation 1-0.2% copper gluconate and 0.1% zinc gluconate with Lactobacillus plantarum and Formulation 2—0.2% copper gluconate and 0.1% zinc gluconate, and control solutions. Red line (linear line) represents the limit of detection (1 Log). Error bars represent standard deviations.

FIG. 19 is a graph showing the average quantities of Staphylococcus aureus recovered from a pre-formed biofilm (72 hour), following 24 hours treatment with Formulation 1-0.2% copper gluconate and 0.1% zinc gluconate with Lactobacillus plantarum and Formulation 2—0.2% copper gluconate and 0.1% zinc gluconate, and control solutions. Red line (linear line) represents the limit of detection (1 Log). Error bars represent standard deviations.

FIG. 20 is a graph showing the average quantities of Pseudomonas aeruginosa recovered from a pre-formed biofilm (72 hour), following 24 hours treatment with Formulation 1-0.2% copper gluconate and 0.1% zinc gluconate with Lactobacillus plantarum and Formulation 2—0.2% copper gluconate and 0.1% zinc gluconate, and control solutions. Red line (linear line) represents the limit of detection (1 Log). Error bars represent standard deviations.

FIG. 21 is a graph showing the average quantities of Candida albicans recovered from a pre-formed biofilm (72 hour), following 72 hours treatment with Formulation 1-0.2% copper gluconate and 0.1% zinc gluconate with Lactobacillus plantarum and Formulation 2-0.2% copper gluconate and 0.1% zinc gluconate, and control solutions. Red line (linear line) represents the limit of detection (1 Log). Error bars represent standard deviations.

### DETAILED DESCRIPTION

Further features of certain embodiments of the present invention are described below. The practice of embodiments of the present invention will employ, unless otherwise indicated, conventional techniques of molecular biology, microbiology, recombinant DNA technology and immunology, which are within the skill of those working in the art.

Most general molecular biology, microbiology recombinant DNA technology and immunological techniques can be found in Sambrook et al, Molecular Cloning, A Laboratory Manual (2001) Cold Harbor-Laboratory Press, Cold Spring Harbor, N.Y. or Ausubel et al., Current protocols in molecular biology (1990) John Wiley and Sons, N.Y. Unless defined otherwise, all technical and scientific terms used herein have the same meaning as commonly understood by one of ordinary skill in the art to which this disclosure belongs. For example, the Concise Dictionary of Biomedicine and Molecular Biology, Juo, Pei-Show, 2nd ed., 2002, CRC Press; The Dictionary of Cell and Molecular Biology, 3rd ed., Academic Press; and the Oxford University Press, provide a person skilled in the art with a general dictionary of many of the terms used in this disclosure.

Units, prefixes and symbols are denoted in their Système International de Unitese (SI) accepted form. Numeric ranges are inclusive of the numbers defining the range.

Aspects of the present invention provide a composition comprising a copper salt and a zinc salt. Aptly, the zinc salt is zinc gluconate and the copper salt is copper gluconate.

Aspects of the present invention provide a combination comprising a copper salt and a zinc salt. Aptly, the zinc salt is zinc gluconate and the copper salt is copper gluconate.

Copper is a chemical element which is biologically essential for most living organisms and is found in all body tissues. Copper plays numerous physiological roles, including, but not limited to, maintenance of bones, blood vessels, nerves, and immune function. Copper also contributes to iron absorption and has an important role in maintaining collagen and elastin, with a further antioxidant function also reported—via acting as a cofactor for the antioxidant enzyme, superoxide dismutase.

Copper gluconate is a copper salt of D-gluconic acid, which helps promote the bioavailability of copper. This soluble copper form is found naturally in a wide variety of foods and can also be taken as a supplement. Copper gluconate is recognized as a GRAS (generally recognized as safe) substance by the US Department of Health and is orally administered as a prophylaxis and a treatment for copper deficiency.

In certain embodiments, the antimicrobial composition comprises copper gluconate.

In certain embodiments, the antimicrobial combination comprises copper gluconate.

Zinc is a chemical element and is the second-most-abundant trace mineral in the body. This trace element acts as a catalytic and structural element in hundreds of metalloproteins, encompassing a range of biological processes from metabolism and digestion to immune and nerve function (to name but a few).

In particular, zinc is critical for efficient cell growth and division, with its presence necessary for gene expression, protein synthesis and DNA synthesis. The role of zinc in immune function, inflammation and collagen synthesis makes it important for healing, with sup-optimal levels associated with delayed wound healing. Therefore, zinc has previously been used in the treatment of skin injuries, burns and ulcers.

The body does not naturally produce zinc, and appropriate levels must be obtained from food or supplements, otherwise detrimental zinc deficiencies can occur. Although uncommon, symptoms of zinc deficiency include, but are not limited to, impaired growth and development, increased risk of infection, skin rashes, chronic diarrhoea, impaired wound healing, and behavioural issues.

Dietary sources of zinc include beans, animal meats, nuts, fish and other seafood, whole grain cereals, and dairy products. There are several zinc supplements commercially available, for instance zinc gluconate, this zinc salt of gluconic acid has been suggested to promote bioavailability. Zinc gluconate is recognized as a GRAS (generally recognized as safe) substance by the US Department of Health and is orally administered as a prophylaxis and a treatment for zinc deficiency.

In certain embodiments, the antimicrobial composition comprises zinc gluconate.

In certain embodiments, the antimicrobial composition comprises copper gluconate and zinc gluconate.

In certain embodiments, the antimicrobial combination comprises zinc gluconate.

In certain embodiments, the antimicrobial combination comprises copper gluconate and zinc gluconate.

Lactobacillus plantarum

As used herein, Lactobacillus plantarum is a lactic acid bacterium. These are Gram-positive, non-spore forming bacteria that grow anaerobically in a large range of environmental niches. L. plantarum is commonly found in the human—and other mammalian—gastrointestinal tracts, saliva, and various food products.

L. plantarum are considered safe probiotics, with reported health benefits of their presence including, but not limited to, reduction of gastrointestinal infection, reduced inflammation reduced risk of inflammatory bowel disease, increased would healing and beneficial modulation of the immune system.

It is considered that L. plantarum may function as a probiotic by secreting antimicrobial compounds, such as bacteriocin, which inhibit the formation of pathogenic gram-positive and gram-negative colonies.

In addition, L. plantarum display a high tolerance to extreme pH and temperatures, for example the bacterium can survive following exposure to hydrochloric acid (pH 2.0) and bile salts.

In certain embodiments, the composition comprises Lactobacillus plantarum.

In certain embodiments, the composition comprises copper gluconate, zinc gluconate and Lactobacillus plantarum.

In certain embodiments, the combination comprises Lactobacillus plantarum.

In certain embodiments, the combination comprises copper gluconate, zinc gluconate and Lactobacillus plantarum.

In certain embodiments, the Lactobacillus plantarum are in the form of viable microorganism.

In certain embodiments, the Lactobacillus plantarum are in the form of lysates.

In certain embodiments, the Lactobacillus plantarum are in the form of tyndallizates.

As described herein, the composition and/or combination may have anti-microbial activity against one or more of the following microbes:
a) Acinetobacter baumannii;
b) Campylobacter coli;
c) Haemophilus influenzae;
d) Helicobacter aeruginosa;
e) Pseudomonas aeruginosa;
f) Salmonella enteritidis;
g) Enterococcus faecium;
h) Neisseria gonorrhoeae;
i) Staphylococcus aureus; and
j) Streptococcus pneumoniae.

In certain embodiments, the composition and/or combination has anti-microbial activity against two or more of a microbe selected from (a) to (j), e.g. three or more, four or more, five or more, six or more, seven or more, eight or more, nine or more or each of (a) to (j).

As described herein, the composition and/or combination may have anti-microbial activity against one or more of the following microbes:
a) Acinetobacter baumannii;
b) Campylobacter coli;
c) Haemophilus influenzae;
d) Helicobacter aeruginosa;
e) Pseudomonas aeruginosa;
f) Salmonella enteritidis;
g) Enterococcus faecium;
h) Neisseria gonorrhoeae;
i) Stomatococcus mucilaginous;
j) Staphylococcus aureus;
k) Staphylococcus hominis;
I) Streptococcus pneumoniae;
m) Streptococcus mutans;
n) Cutibacterium;
o) Anaerococcus;
p) Peptoniphilus;
q) Candida albicans;
r) Malassezia restricta;
s) Malassezia globosa;
t) Propionibacterium;
u) Gardnerella vaginalis;
v) Escherichia coli;
w) Prevotella;
x) Peptostreptococcus;
y) Bacteroides; and
z) Corynebacterium.

In certain embodiments, the composition and/or combination has anti-microbial activity against two or more of a microbe selected from (a) to (z), e.g. three or more, four or more, five or more, six or more, seven or more, eight or more, nine or more or each of (a) to (z).

Bacteria

The Gram-stain test was developed by bacteriologist Hans Gram to distinguish different types of bacteria based on their cell wall constituents. Gram staining involves three steps: staining with a crystal violet dye, decolorization, and counterstaining. The theory behind the test is that Gram-positive bacteria retain the crystal violet stain during the decolorization process due to a thicker peptidoglycan layer in the cell membrane, whereas Gram negative bacteria lose the crystal violet stain.

As used herein Gram-positive bacteria are bacteria which give a positive result in the Gram stain test. Typically, a positive result is characteristic of bacteria that have a cell wall composed of a thick peptidoglycan layer and no outer lipid membrane. Examples of Gram-positive bacteria include, but are not limited to, Staphylococcus aureus, Streptococcus pneumoniae, Stomatococcus mucilaginous, Streptococcus mutans, Staphylococcus hominis, Staphylococcus epidermidis, Mycobacterium tuberculosis, Bacillus cereus, Micrococcus luteus, Enterococcus faecalis, Peptostreptococcus, Cutibacterium, Corynebacterium, Anaerococcus, Corynebacterium, Peptoniphilus and Clostridium perfringens.

As used herein Staphylococcus aureus is a gram-positive coccus and is a facultative anaerobic member of the micrococcaceae family. S. aureus is present in human's gut microflora and colonizes skin, but may become pathogenic causing a variety of ailments from minor skin infections and abscesses, to life-threatening diseases, including but not limited to, pneumonia, meningitis and septicaemia. The pathogenicity of S. aureus is due to the toxins produced by the bacteria, aided by its evasiveness to the immune response and antibiotic resistance.

In certain embodiments, compositions and combinations provided by the present invention are effective antimicrobials against bacteria.

In certain embodiments, compositions and combinations provided by the present invention are effective antimicrobials against Gram-positive bacteria. In further embodiments compositions and combinations provided by the present invention are effective antimicrobials against Staphylococcus aureus.

As used herein Gram-negative bacteria are bacteria which give a negative result in the Gram stain test. Typically, a negative result is characteristic of bacteria that have a cell wall composed of a thin peptidoglycan layer and an outer lipid membrane. Examples of Gram-negative bacteria include, but are not limited to, Pseudomonas aeruginosa, Escherichia coli, Klebsiella pneumoniae, Haemophilus influenzae, Neisseria gonorrhoeae, Neisseria meningitidis, Helicobacter pylori, Prevotella spp, Bacteroides spp and Chlamydia trachomatis.

As used herein, Pseudomonas aeruginosa is a common Gram-negative bacillus which acts as an opportunistic pathogen, with almost all clinical infections associated with a compromised host defence. The three most prevalent pathologies following P. aeruginosa infection are bacteremia in severe burn victims; chronic lung infection in cystic fibrosis patients and acute ulcerative keratitis in users of extended-wear contact lenses. The pathogenicity of P. aeruginosa is due to the secretion of virulence factors including, but not limited to, pili, flagella, lipopolysaccharide, proteases, quorum sensing and exotoxin A.

In certain embodiments, compositions provided by the present invention are effective antimicrobials against Gram-negative bacteria. In further embodiments the compositions provided by the present invention are effective antimicrobials against Pseudomonas aeruginosa.

As used herein, Gardnerella vaginalis is a Gram-variable bacteria, this pleomorphic bacteria is a facultative nonmotile anaerobic coccobacilli. Gardnerella vaginalis does not form part of the normal vaginal flora, with the presence of Gardnerella vaginalis disrupting the normal vaginal flora and so causing bacterial vaginosis.

Fungi

As used herein, Fungi are heterotrophic eukaryotes that are traditionally classified morphologically into yeast and filamentous hyphae forms. Fungi are highly adaptable and can colonize almost every niche within their host, establishing symbiotic, commensal, latent or pathogenic relationships with plants and animals. The human gut microflora comprises numerous species of fungi, however pathogenic fungi can also invade the body following inhalation of spores or small yeast cells. Examples of fungal genus include, but are not limited to, Aspergillus, Alternaria, Candida, Cladosporium, Malassezia and Penicillium.

As used herein, Candida albicans is a polymorphic fungus, able to grow as unicellular budding yeast or in filamentous pseudohyphal and hyphal forms. Typically, C. albicans is a benign member of the mucosal flora, however this usually commensal fungus can become pathogenic in certain contexts causing mucosal infections. Further if the hyphal form of C albicans penetrates the endothelia allowing access to the bloodstream, a life-threatening infection known as candidaemia can ensue.

As used herein, Candida auris is the most common fungal pathogen worldwide, this multidrug-resistant yeast has developed an extensive repertoire of virulent mechanisms that enable effective colonization and infection of the host under suitable conditions. This opportunistic human fungal pathogen causes a range of infections in the bloodstream, gut, wounds, and other sites. Some infections are resistant to all antifungal drugs and so do not respond to treatment, C. auris is responsible for around 7% of hospital-acquired infections in the United States.

As used herein, Aspergillus brasiliensis is the most common species of the genus Aspergillus. It is ubiquitous in soil and is a frequent food contaminant. Human diseases caused by A. brasiliensis are rare compared to other members of the genus. However, A. brasiliensis can cause pulmonary infections in weakened immune systems, and in rare cases can form a ball of matted hyphae called a aspergilloma.

As used herein, Malassezia is a genus of fungi which forms part of the human cutaneous commensal flora. Malassezia species have been associated with a wide range of dermatological disorders including, but not limited to, dandruff and seborrheic dermatitis. For example, Malassezia restricta and Malassezia globosa are both commonly found on healthy human skin, but are also deemed opportunistic pathogens involved in skin disorders including, but not limited to, dandruff and seborrheic dermatitis.

In certain embodiments, compositions and combinations provided by the present invention are effective antimicrobials against fungi.

In further embodiments the compositions and combinations provided by the present invention are effective antimicrobials against Aspergillus, Alternaria, Candida, Cladosporium, Malassezia and Penicillium fungi. In some embodiments, the compositions provided by the present invention are effective antimicrobials against Candida albicans, Candida auris and Aspergillus brasiliensis.

In further embodiments the composition and/or combination provided by the present invention is an effective antimicrobial against polymorphic fungi, including but not limited to, Candida albicans.

Not only can antimicrobial compositions and combinations be utilised for the treatment of infections, but such compositions can also be used in the prevention of infections, which plays an important role in health care. In certain embodiments, antimicrobial compositions provided by the present invention may be used in a prophylactic treatment to prevent infection.

As described herein the term “genital tract infection” refers to any infection of the genital tract. The three main causes of infection in the female genital tract include: a) sexually transmitted diseases (STDs), examples include, but are not limited to: chlamydia, gonorrhoea, chancroid, and human immunodeficiency virus (HIV); b) endogenous infections, which are caused by dysbiosis or shifts in the microbiome resulting in overgrowth of the organisms normally present in the genital tract of healthy women, examples include, but are not limited to bacterial vaginosis and vulvovaginal candidiasis; and c) iatrogenic infections, which are associated with improperly performed medical procedures, examples include, but are not limited to unsafe abortion or poor delivery practices. In certain embodiments, the composition and/or combination is for the treatment of a genital tract infection. Aptly, the genital tract infection is selected from bacterial vaginosis and vulvovaginal candidiasis.

Once established, infections can ascend the female reproductive tract, and cause inflammation and subsequent scarring, thus female genital tract infections are a common cause of reduced fecundability and sterility.

Lactobacillus species is one of the dominant vaginal bacteria in healthy asymptomatic women, via the production of lactic acid and hydrogen peroxide these bacteria inhibit the growth of numerous pathogenic micro-organisms.

In certain embodiments, the antimicrobial compositions provided by the present invention may be used to treat and/or prevent infections. Aptly, the antimicrobial composition and/or combination may be used to treat and/or prevent infections at any anatomic location and/or organ system, particularly the skin and mucosa. In further embodiments, the infection may be in the female genital tract.

In certain embodiments the micro-organisms causing genital infections include, but are not limited to, Gardnerella vaginalis, Escherichia coli, Prevotella, Peptostreptococcus, Bacteroides spp and Corynebacterium.

In certain embodiments the compositions and/or combinations provided by the present invention are effective antimicrobials against Gardnerella vaginalis, Escherichia coli, Prevotella, Peptostreptococcus, Bacteroides spp and Corynebacterium.

In certain embodiments, the composition and/or combination is for use in the treatment of an infection in a subject of one or more of the following bacterial organisms:
a) Gardnerella vaginalis;
b) Escherichia coli;
c) Prevotella;
d) Peptostreptococcus;
e) Bacteroides spp; and
f) Corynebacterium.

In certain embodiments compositions and/or combinations provided by the present invention are effective antimicrobials against genital infections.

In certain embodiments, the composition may be in the form of a cream, a tablet and/or a pessary In certain embodiments, the composition may be a gel, a lotion, a paste and/or may be configured to form a physical barrier.

In certain embodiments the antimicrobial compositions provided by the present invention have a protective, restoring effect on the microbiome.

As described herein, the compositions and/or combinations of certain embodiments of the present invention may be used to treat wounds e.g. to prevent microbial infection of wounds or to reduce microbial infection of a wound.

As used herein the term “wound” refers to any damage or break in the surface of the skin. Types of wound include, but are not limited to, abrasions, incisions, lacerations, punctures, avulsions, ulcers (originating from pressure, venous stasis, or diabetes mellitus) and burns. In certain embodiments, the composition may be used to treat a chronic wound. As used herein the term “chronic wound” refers to a wound that has failed to progress through the phases of healing in an orderly and timely fashion. Risk factors for developing a chronic wound include, but are not limited to, diabetes, obesity, age, decreased cardiovascular function, decreased mobility, previous scar tissue, tissue hypoxia from pressure or anaemia, infection and circulation or clotting disorders.

In certain embodiments, the composition and/or combination may be for use to treat a wound in combination with a further wound treatment. For example, the composition may be for use in combination with an antibiotic treatment, debridement (which is the medical removal of dead, damaged, or infected tissue), vacuum-assisted closure, oxygenation, moist wound healing, dressings and bandages, removing mechanical stress (sometimes referred to as off-loading), adding cells or other materials to secrete (or enhance the levels of) healing promoting factors, pneumatic compression device, UV-C therapy and hyperbaric oxygen therapy (HBOT).

In certain embodiments, the composition and/or combination is provided in a wound dressing and/or bandage. Generally, dressings and bandages can be separated into two categories, passive and active. The passive wound dressings serve only to provide mechanical protection and/or a barrier to infection, hence passive dressings do not comprise an active therapeutic ingredient, but rather protect the wound site facilitating spontaneous wound healing. Examples of passive dressings include, but are not limited to, gauze and adhesive bandages.

Conversely, active dressings and bandages comprise one or more biologically active compound, which can act at the site of a wound to promote wound healing. For instance, one type of active dressing may deliver or have been impregnated with antimicrobials to effectively reduce the pathogenic microbial load at a wound site.

In certain embodiments the compositions provided by the present invention are antimicrobial compositions. In further embodiments the antimicrobial compositions may form part of an active dressing.

In certain embodiments the composition and/or combinations provided by the present invention comprises copper gluconate, zinc gluconate and any appropriate probiotic micro-organism. As used herein probiotics are live microorganisms, lysates thereof and/or tyndallizates thereof, which when administered in adequate amounts confer a health benefit on the host. Examples of probiotic micro-organisms include, but are not limited to, Bacillus coagulans, Bifidobacterium bifidum, Bifidobacterium infantis, Bifidobacterium thermophilus, Bifidobacterium longum, Bifidobacterium animalis, Enterobacter faecium, Enterobacter faecalis, Lactobacillus acidophilus, Lactobacillus brevis, Lactobacillus casei, Lactobacillus bulgaricus, Lactobacillus cellobiosus, Lactobacillus plantarum, Lactobacillus rhamnosus GG, Lactobacillus delbrueckii, Lactobacillus fermentum, Leuconostoc mesenteroides, Pediococcus acidilactici, Saccharomyces boulardii, Saccharomyces cerevisiae, Saccharomyces carlsbergensis, Streptococcus lactis, Streptococcus thermophilus, Streptococcus cremonis and Streptococcus alivarius. In further embodiments, the probiotic micro-organism is Lactobacillus plantarum.

As used herein, the term “lysates” refers to a preparation of microbial cell extracts produced by lysis of microorganisms. Such cell lysis may be achieved by any suitable cell disruption method known in the art, including, but not limited to, grinding, mechanical disruption, freeze-thaw cycles, liquid homogenization, sonication, or by the use of chemicals including, but not limited to, organic solvents, chelating agents, detergents or surfactants and chaotropic agents. Following the lysis of probiotic cells, the probiotic lysates produced can be used as prebiotics.

As used herein, the term “tyndallizates” refers to heat-treated microorganisms, for example, probiotic microorganisms that have undergone tyndallization, which is a process of sterilizing by repeated boiling. Following the process of tyndallization, the resulting tyndallizates may comprise cell fractions and supernatants.

In some embodiments the composition provided by the present invention comprises copper gluconate and zinc gluconate.

In some embodiments, the composition and/or combination further comprises one or more probiotic bacteria and/or yeast. In some embodiments, the composition further comprises a probiotic bacteria selected from Lactobacillus acidophilus, Lactobacillus casei, and Lactobacillus rhamnosus and combinations thereof. In some embodiments, the composition and/or combination comprises Bifidobacterium longum alone or in combination with one or more Lactobacillus acidophilus, Lactobacillus casei, and Lactobacillus rhamnosus.

In some embodiments, the composition comprises E. faecium.

In some embodiments, the combination comprises E. faecium.

In some embodiments, the combination may be for topical application. In some embodiments, the composition may be for topical application. As used herein topical compositions are compositions for the topical delivery of substances such as pharmaceutical agents. The topical composition may be applied to the skin and/or mucosa of a subject, e.g. a human, to aid in treatment of medical conditions or diseases, and/or the symptoms associated thereof. Aptly, the composition may be in the form of an ointment, a cream and/or a gel. In certain embodiments, the composition is in the form of a pressed tablet, e.g. a pessary.

In some instances, topical delivery enables the minimum dosage of pharmaceutical agents to be delivered directly to the affected area, providing effective levels of medication whilst limiting side effects. The effective dosage of the pharmaceutical agent delivered topically may be lower than the effective dosage of the pharmaceutical agent taken orally.

The topical composition may further comprise one or more known pharmaceutically acceptable additives, e.g. thickening agents including, but not limited to, carboxy vinyl polymer, hydroxyethyl cellulose, hydroxypropyl cellulose, methyl cellulose, hydroxypropyl methyl cellulose, carboxymethyl cellulose, and salts thereof, carriers, excipients or diluents, unless the incorporation thereof would adversely affect the intended purpose of the present invention.

In certain embodiments, the invention provides a topical composition for the treatment of an infection and/or wound.

In further embodiments the invention provides an antimicrobial composition for direct topical application to a wound site and/or site of infection.

Accordingly, in certain embodiments, a composition and/or combination may be topically applied to a specific location of the body, e.g., the site of infection or the wound site. In further embodiments, a composition provided by the present invention may be topically applied to the skin and/or mucosa. Also, in certain cases, a composition as described herein may be used in the preparation of a medicament for treatment of a wound.

In certain embodiments, the composition is for the treatment of a genital disorder e.g. a genital infection.

In certain embodiments, the composition is for the prevention of a genital disorder e.g. a genital infection. In certain embodiments, the composition is for the treatment and/or prevention of a vaginal disorder. The vaginal disorder may be for example a vaginal infection. Aptly, the patient is a female patient. In certain embodiments, the composition is in the form of a pressed tablet, e.g. a pessary. In certain embodiments, the composition is in the form of a cream, a gel and/or an ointment.

In an aspect of the present invention, there is provided a method of administering a therapeutically effective amount of the antimicrobial composition and/or combination of certain aspects described herein to a patient.

In certain embodiments, the composition and/or combination may be for parenteral administration. The term “parenteral administration” as used herein includes any form of administration in which the compound is absorbed into the subject without involving absorption via the intestines. Exemplary parenteral administrations that are used in the present invention include, but are not limited to intramuscular, intravenous, intraperitoneal, intraoccular, or intraarticular administration. Yet further, parenteral administration also includes administration into a surgical field.

In certain embodiments, the composition and/or combination is for oral administration to a subject e.g. a human subject. Aptly, the composition and/or combination may be comprised in an oral liquid, a tablet, a capsule, an oral paste and/or a chewable product. In certain embodiments, the composition is comprised in a chewable product.

As used herein, the term “chewable product” may refer to a product comprising the composition and/or combination which is processed by a subject by chewing to facilitate release of the active ingredient(s) in the oral cavity. Aptly chewable products may act as a transport medium to enable the oral ingestion of the active ingredients. Aptly chewable products may rapidly soften or disintegrate following exposure to saliva. Such chewable products provide an alternative for people who have difficulty swallowing whole capsules or tablets.

In certain embodiments the composition and/or combination may be for oral administration. In certain embodiments the composition and/or combination is in the form of a chewable product.

In certain embodiments the chewable product comprises zinc and copper formulations.

In certain embodiments the chewable product comprises zinc and copper formulations in combination with one or more probiotics. Aptly probiotics may be one or more of any live probiotic microorganism, lysate thereof or tyndallizate thereof. Aptly the probiotic microorganisms include, but are not limited to, Bacillus coagulans, Bifidobacterium bifidum, Bifidobacterium infantis, Bifidobacterium thermophilus, Bifidobacterium longum, Bifidobacterium animalis, Enterobacter faecium, Enterobacter faecalis, Lactobacillus acidophilus, Lactobacillus brevis, Lactobacillus casei, Lactobacillus bulgaricus, Lactobacillus cellobiosus, Lactobacillus plantarum, Lactobacillus rhamnosus GG, Lactobacillus delbrueckii, Lactobacillus fermentum, Leuconostoc mesenteroides, Pediococcus acidilactici, Saccharomyces boulardii, Saccharomyces cerevisiae, Saccharomyces carlsbergensis, Streptococcus lactis, Streptococcus thermophilus, Streptococcus cremonis and Streptococcus alivarius.

In some embodiments the chewable product comprises zinc and copper formulations with multiple probiotics. Aptly probiotics may be two or more of any live probiotic microorganism, lysate thereof or tyndallizate thereof. Aptly the probiotic microorganisms include, but are not limited to, Bacillus coagulans, Bifidobacterium bifidum, Bifidobacterium infantis, Bifidobacterium thermophilus, Bifidobacterium longum, Bifidobacterium animalis, Enterobacter faecium, Enterobacter faecalis, Lactobacillus acidophilus, Lactobacillus brevis, Lactobacillus casei, Lactobacillus bulgaricus, Lactobacillus cellobiosus, Lactobacillus plantarum, Lactobacillus rhamnosus GG, Lactobacillus delbrueckii, Lactobacillus fermentum, Leuconostoc mesenteroides, Pediococcus acidilactici, Saccharomyces boulardii, Saccharomyces cerevisiae, Saccharomyces carlsbergensis, Streptococcus lactis, Streptococcus thermophilus, Streptococcus cremonis and Streptococcus alivarius.

In certain embodiments, the chewable product may be an effective antimicrobial in promoting total oral hygiene.

In certain embodiments, the chewable product may be for use in the treatment and/or prevention of halitosis.

In certain embodiments, the chewable product may be for use in the treatment and/or prevention of dental caries.

A “patient” or “subject” includes any human or other mammalian subject that receives either prophylactic or therapeutic treatment. Treatment is considered prophylactic if administered to an individual susceptible to, or otherwise at risk of infection and/or wound infection. Treatment is considered therapeutic if administered to an individual suspected of having, or already suffering from an infection for example but not limited to a wound infection.

In certain embodiments, the composition and/or combination is for use in the treatment and/or prevention of a microbial infection of a subject by one or more of the following micro-organisms:
a) Acinetobacter baumannii;
b) Campylobacter coli;
c) Haemophilus influenzae;
d) Helicobacter aeruginosa;
e) Pseudomonas aeruginosa;
f) Salmonella enteritidis;
g) Enterococcus faecium;
h) Neisseria gonorrhoeae;
i) Staphylococcus aureus; and
j) Streptococcus pneumoniae.

The infection may be a systemic infection.

Thus, in certain embodiments, the composition may be for oral administration and/or parenteral administration.

Thus, in certain embodiments, the combination may be for oral administration and/or parenteral administration.

Compositions of certain embodiments of the invention may be administered with or without an excipient. Excipients include, for example, encapsulating materials or additives such as absorption accelerators, antioxidants, binders, buffers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents and mixtures thereof. The excipients may be pharmaceutically acceptable excipients.

Excipients for preparation of compositions comprising a compositions of certain embodiments of the invention to be administered orally in solid dosage form include, for example, agar, alginic acid, aluminum hydroxide, benzyl alcohol, benzyl benzoate, 1,3-butylene glycol, carbomers, castor oil, cellulose, cellulose acetate, cocoa butter, corn starch, com oil, cottonseed oil, cross-povidone, diglycerides, ethanol, ethyl cellulose, ethyl laureate, ethyl oleate, fatty acid esters, gelatin, germ oil, glucose, glycerol, groundnut oil, hydroxypropylmethyl cellulose, isopropanol, isotonic saline, lactose, magnesium hydroxide, magnesium stearate, malt, mannitol, monoglycerides, olive oil, peanut oil, potassium phosphate salts, potato starch, povidone, propylene glycol, Ringer's solution, safflower oil, sesame oil, sodium carboxymethyl cellulose, sodium phosphate salts, sodium lauryl sulfate, sodium sorbitol, soybean oil, stearic acids, stearyl fumarate, sucrose, surfactants, talc, tragacanth, tetrahydrofurfuryl alcohol, triglycerides, water, and mixtures thereof.

Excipients for preparation of compositions of certain embodiments of the present invention to be administered orally in liquid dosage forms include, for example, 1,3-butylene glycol, castor oil, com oil, cottonseed oil, ethanol, fatty acid esters of sorbitan, germ oil, groundnut oil, glycerol, isopropanol, olive oil, polyethylene glycols, propylene glycol, sesame oil, water and mixtures thereof.

Excipients for preparation of compositions of certain embodiments of the present invention to be administered osmotically include, for example, chlorofluorohydrocarbons, ethanol, water and mixtures thereof. Excipients for preparation of compositions of certain embodiments of the present invention to be administered parenterally include, for example, 1,3-butanediol, castor oil, com oil, cottonseed oil, dextrose, germ oil, groundnut oil, liposomes, oleic acid, olive oil, peanut oil, Ringer's solution, safflower oil, sesame oil, soybean oil, U.S.P. or isotonic sodium chloride solution, water and mixtures thereof.

Excipients for preparation of compositions comprising a compound of this invention to be administered rectally or vaginally include, for example, cocoa butter, polyethylene glycol, wax and mixtures thereof.

Aptly, the composition is for administration in a therapeutically effective amount. As used herein, the term “therapeutically effective amount”” is taken to refer to an amount of an agent, e.g. an agent of certain embodiments of the present invention that produces a desired therapeutic effect in the patient, such as preventing or treating a target condition or alleviating symptoms associated with the condition. Aptly, the precise therapeutically effective amount is an amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. A person skilled in the clinical and pharmacological arts will be able to determine a therapeutically effective amount of the composition to administer through routine experimentation, namely by monitoring a patient's response to administration of a compound and adjusting the dosage accordingly. For additional guidance, see Remington: The Science and Practice of Pharmacy 21st Edition, Univ. of Sciences in Philadelphia (USIP), Lippincott Williams & Wilkins, Philadelphia, Pa., 2005.

AATCC 100 Test Method

As used herein, the AATCC 100 Test Method was developed by the American Association of Textile Chemists and Colorists to assess the antibacterial finishes on textile materials. This quantitative antimicrobial test is the industry standard, and typically evaluates the antibacterial properties of textiles over a 24-hour contact period. The method quantitatively assesses bacteriostatic (growth inhibition) or bactericidal (killing of bacteria) properties, facilitating continuity in approaches and replicability of results via calculating the reduction of bacterial growth compared to a negative control.

The AATCC 100 method generally involves the steps of: sample preparation, sterilisation, inoculation, incubation, washing/shaking out, followed by counting. However, this method can be modified to enable a variety of antimicrobial testing protocols to be carried out, using the principles of the AATCC 100 test method.

Aptly, the antimicrobial capability of any compound or composition in relation to any specific micro-organism, may be assessed by any means known in the art, non-limiting examples may include survival rate testing and AATCC 100 test method protocols.

Biofilm

Typically, micro-organisms (particularly bacteria) prefer existence as a surface-attached community—as opposed to life as free-floating individuals—and predominantly occur in a biofilm phenotype (also referred to as slime), in which surface-adhering bacteria are encased within an extracellular polymeric substance. The development of a biofilm may allow formation of aggregated cell colonies, which can protect microbes from some antimicrobial agents and increase antibiotic resistance. Therefore, eliminating bacteria and fungi within the biofilm reservoir can be difficult to achieve.

As used herein, a biofilm is an assemblage of surface-associated microbial cells that are enclosed in an extracellular polymeric substance matrix. Research using animal models has indicated that the presence of biofilm in a wound produces a low-grade but persistent inflammatory response, which impairs both epithelialization and granulation tissue formation (Gurjala & Geringer, 2011). The capacity of biofilm formation in wounds is well established, with some even suggesting that as many as half of all chronic wounds could contain a biofilm (Metcalf & Bowler, 2013).

In certain embodiments compositions and/or combinations provided by the present invention are effective antimicrobials against biofilms.

In further embodiments, compositions and/or combinations provided by the present invention are effective antimicrobials against a Candida albicans biofilm, Pseudomonas aeruginosa biofilm and/or Staphylococcus aureus biofilm.

As used herein, the term dental caries (or tooth decay) refers to a biofilm-mediated disease, wherein the localised destruction of dental hard tissue is caused by demineralisation. This demineralisation of dental hard tissue is a result of local pH levels being lowered by bacterial fermentation of dietary carbohydrates, which creates acidic by-products. Such biofilm forming, dental caries inducing micro-organisms include, but are not limited to, Streptococcus spp. (e.g., Streptococcus mutans), Enterococcus spp. (e.g., Enterococcus faecalis) and/or Candida albicans.

In certain embodiments the compositions and/or combinations provided by the present invention are effective antimicrobials against Streptococcus spp. (e.g., Streptococcus mutans), Enterococcus spp. (e.g., Enterococcus faecalis) and/or Candida albicans.

In certain embodiments, the composition and/or combination is for use in reducing in a subject the microbial load of one or more of the following micro-organisms:
a) Streptococcus spp. (Streptococcus mutans);
b) Enterococcus spp. (Enterococcus faecalis); and
c) Candida albicans.

In certain embodiments compositions and/or combinations provided by the present invention are effective antimicrobials in preventing dental caries.

In certain embodiments the compositions and/or combinations provided by the present invention may be for use in the prevention and/or reduction of body odour. As used herein, the term body odour refers to unpleasant smell on a person's body. Body odour is produced by bacterial transformation of odourless precursor molecules secreted onto the surface of the skin by apocrine glands. In particular, the apocrine glands secrete specific precursor molecules that are odourless, however following the action of bacterial enzymes, volatile odourants are released from the precursors—leading to the production of pungent odour. Therefore, the bacterial microbiome of the epidermis, sweat glands, sweat pores and hair follicles plays an important role in the development of body odour. Such odour-forming micro-organisms include, but are not limited to, Staphylococcus hominis and other Staphylococcus strains, Cutibacterium (formerly Propionibacterium) and Corynebacterium, Anaerococcus and Peptoniphilus species.

In certain embodiments the compositions and/or combinations provided by the present invention are effective antimicrobials against Staphylococcus hominis (and/or other Staphylococcus strains), Cutibacterium, Corynebacterium, Anaerococcus and/or Peptoniphilus species.

In certain embodiments, the composition and/or combination is for use in reducing in a subject the microbial load of one or more of the following micro-organisms:
a) Staphylococcus hominis;
b) other Staphylococcus strains;
c) Cutibacterium;
d) Corynebacterium;
e) Anaerococcus; and
f) Peptoniphilus.

In certain embodiments compositions and/or combinations provided by the present invention are effective antimicrobials in the treatment and/or prevention of body odour.

As used herein, dandruff refers to an unpleasant scalp disorder involving itchy, flaking skin. The most common cause of dandruff is generally thought to be microbial. In particular, the lipophilic yeast, Malassezia—which is the major fungi colonising the human scalp—is thought to induce inflammation on the epidermis via oleic acid. Such dandruff causing fungi include, but are not limited to, Malassezia restricta and Malassezia globosa. Furthermore, recent research has demonstrated the importance of scalp-colonising bacteria in dandruff aetiology. Such dandruff causing bacteria include, but are not limited to, Propionibacterium.

In certain embodiments the compositions and/or combinations provided by the present invention are effective antimicrobials against Malassezia restricta, Malassezia globosa and Propionibacterium.

In certain embodiments, the composition and/or combination is for use in reducing in a subject the microbial load of one or more of the following micro-organisms:
a) Malassezia restricta;
b) Malassezia globosa; and
c) Propionibacterium.

In certain embodiments compositions and/or combinations provided by the present invention are effective antimicrobials in the treatment and/or prevention of dandruff.

As used herein, halitosis, or bad breath, refers to an oral malodour. Halitosis is associated with the resident microbial flora in the oral cavity, which are able to produce volatile sulphur compounds. For example, anaerobic oral microorganisms produce volatile sulphur compounds in the proteolytic degradation process of peptides.

Such halitosis causing microorganisms include, but are not limited to, Stomatococcus mucilaginous, Streptococcus spp. (e.g., Streptococcus mutans), Enterococcus spp. (e.g., Enterococcus faecalis) and/or Candida albicans.

In certain embodiments the compositions and/or combinations provided by the present invention are effective antimicrobials against Stomatococcus mucilaginous, Streptococcus spp. (e.g., Streptococcus mutans), Enterococcus spp. (e.g., Enterococcus faecalis) and/or Candida albicans.

In certain embodiments, the composition and/or combination is for use in reducing in a subject the microbial load of one or more of the following micro-organisms:
a) Stomatococcus mucilaginous
b) Streptococcus spp. (Streptococcus mutans);
c) Enterococcus spp. (Enterococcus faecalis); and
d) Candida albicans.

In certain embodiments compositions and/or combinations provided by the present invention are effective antimicrobials in the treatment and/or prevention of halitosis.

In certain aspects of the present invention there is provided antibacterial compositions in the form of cleaning products, washes, surface coatings or other compositions which are not for medical treatment of the human or animal body.

Such agents may be useful for removing, killing, or preventing the accumulation of bacteria on a surface, or inhibiting the action or growth of the bacteria.

Anti-bacterial compositions according to the invention may be useful for treating biomaterials, implants and prosthesis (including stents, valves, eyes, hearing aids, gastric bands, dentures, artificial joint replacements etc), surgical instruments or other medical devices prior to administration to, or treatment of, or use with, a patient or subject. The antibacterial compositions may be useful for treating surfaces prone to colonisation or exposure to bacterial, such as handrails, food preparation surfaces, kitchen surfaces or equipment, tables, sinks, toilets or other bathroom hardware.

Antibacterial compositions may comprise agents in addition to the lysate, such as cleaning agents, stabilisers, anionic surfactants, perfumes, chelating agents, acids, alkalis, buffers or detergents. Such agents may facilitate or enhance the antibacterial properties of the agent, such as killing or inhibiting bacteria, or preventing the recolonisation of the cleaned surface. Therefore, said compositions could be beneficial in the treatment and/or prevention of numerous disorders and conditions including, but not limited to: psoriasis, icthyosis, dermatitis, wound healing, acne (including Hidradenitis Suppurativa), Burns, Diaper Rash, Netherton's Syndrome, Actinic Keratosis, Dermatomycoses, Dermatosis or Ectodermal Dysplasia.

Furthermore, said compositions could be beneficial in the treatment and/or prevention of numerous disorders and conditions including, but not limited to: body odour, psoriasis, icthyosis, dermatitis, atopic dermatitis, dandruff, dental caries, halitosis, genital infections, wound healing, acne (including Hidradenitis Suppurativa), Rosacea, Burns, Diaper Rash, Netherton's Syndrome, Actinic Keratosis, Dermatomycoses, Dermatosis and/or Ectodermal Dysplasia.

Our initial observations indicate that the combinations e.g. those comprising probiotics described herein could be applied to a number of applications in skin care that include:

wound healing, coating technology for wound dressings, improved skin barrier creams for cosmetic and therapeutic uses (e.g. atopic dermatitis); antibacterial products such as hand washes and soaps, and cosmetic preparations aimed at maintenance/repair of the skin's barrier function and treatments to restore a damaged barrier due to sun exposure and ageing.

### Examples

In the following, the invention will be explained in more detail by means of non-limiting examples of specific embodiments. In the example experiments, standard reagents and buffers free from contamination are used.

Materials and Methods

Test Microorganism:

Gram-Negative Bacteria
Acinetobacter baumannii (NCTC® 13301™)
Campylobacter coli (ATCC® BAA-371™)
Enterobacter cloacae (NCTC® 13464™)
Haemophilus influenzae (NCTC® 12699™)
Helicobacter pylori (NCTC® 1320™)
Pseudomonas aeruginosa (NCTC® 13437™ and NCIMB 10434)
Salmonella enteritidis (NCTC® 13349™)

Gram-Positive Bacteria
Lactobacillus plantarum (ATCC® 8014™)
Enterococcus faecium ((VRE) ATCC® 700221™)
Neisseria gonorrhoeae (NCTC® 13480™)
Staphylococcus aureus (NCTC® 13142™ and ATCC© 6538™)
Streptococcus pneumoniae (ATCC® BAA-343™)

Fungi
Candida albicans (ATCC© 90028™ and ATCC® MYA 2876™)
Candida auris (NCPF© 8971™)
Aspergillus brasiliensis (ATCC© 16404™)

Test Agents

TABLE 1
Test agents used throughout the specification. PBS = phosphate
buffered saline, MRSB = de Man, Rogosa and Sharpe broth.
Lactobacillus plantarum ATCC ® 8014 ™ cultures in Formulation
1 were prepared to a concentration of 1 × 10⁸ ± 5 ×
10⁷ CFUmL⁻¹.

| Test agent | Test agent format | Lot number |
|---|---|---|
| Copper gluconate | Test liquid | STBG3160V |
| Zinc gluconate | Test liquid | 10208050 |
| Copper gluconate | Test liquid | STBG3160V/ |
| and zinc gluconate | | 10208050 |
| Formulation 1 | 0.2% Copper gluconate and | STBG3160V, |
| | 0.1% Zinc gluconate with | 10208050 |
| | Lactobacillus plantarum | |
| Formulation 2 | 0.2% Copper gluconate and | STBG3160V, |
| | 0.1% Zinc gluconate without | 10208050 |
| | Lactobacillus plantarum | |
| Positive control | Bleach based product | 9 161 2696 |
| Negative control | PBS + 1% MRSB | PBS200103, |
| | | TSB200103, |
| | | BHIB 200122, |
| | | TSBHB 200227, |
| | | PBS191107, |
| | | MRSB191031 |

Equipment and Media

Equipment:
Calibrated balance—A&D Instruments, UK
CO₂ microbiological incubator (37° C., 30° C. and 25° C.)—ThermoFisher Scientific, UK or SciQuip, UK
CDC Biofilm Reactor®— Biosurface Technologies, USA
Polycarbonate coupon, RD128-PC—Biosurface Technologies, USA
Shaking incubator—Innova 4300, Eppendorf UK Limited
Sonic water bath—VWR, UK
UKAS calibrated pipettes (0.5-1000 μL range)—Proline® Plus, Sartorius, UK
UKAS calibrated multichannel pipettes (P300 and P20)—Gilson®, UK, Scientific Laboratory Supplies (SLS), UK
96 well plates—Scientific Laboratory Supplies (SLS), UK
Sterile universal container— SLS, UK

Media:
Sabouraud Dextrose Agar (SDA), Acumedia®— SLS, UK
Sabouraud Dextrose Broth (SDB), Acumedia®— Trafalgar Scientific, UK
Tryptone Soya Broth (TSB), Acumedia®— Trafalgar Scientific, UK
Tryptone Soya Agar (TSA)—Southern Group Laboratories, UK
Brain Heart Infusion Agar, Acumedia®— Trafalgar Scientific, UK
Brain Heart Infusion Broth, Acumedia®— Trafalgar Scientific, UK
Phosphate Buffered Saline (PBS)—SLS, UK
Media specific for WHO priority pathogens
Fetal Calf Serum (heat inactivated)—Gibco, UK
Fetal Bovine Serum—SLS, UK
Defibrinated horse blood—BioTechne, UK
Peptone—Sigma Aldrich, UK
Sodium Chloride—Acros Organics, UK
de Man, Rogosa and Sharpe Agar (MRSA)—SLS, UK
de Man, Rogosa and Sharpe Broth (MRSB)—SLS, UK
Dey-Engley Neutraliser (DE-N)—Acumedia®, Trafalgar Scientific, UK
Quench neutraliser—Tween(80) 30 g, Lecithin 3 g, Histidine 1 g, Sodium thiosulphate 4 g, pH 7 Buffer 20 mL, and Distilled Water 1 L
Deionised water

Methods

Phase 1: Neutraliser Validation

A selection of neutralisers were assessed for their efficacy and toxicity against Lactobacillus plantarum in triplicate. A neutraliser was selected if it was effective in stopping the activity of the test product and was not toxic to the microorganism tested.

Two grams of copper gluconate and 1.5 g of zinc gluconate were vortexed in universal tubes containing 10 mL of deionised water until fully dissolved. These were identified as the maximum amounts that were 100% soluble in 10 mL deionised water at 37° C.±2° C. Five millilitres of each solution were mixed to create a 10 mL copper gluconate and zinc gluconate solution. One millilitre of each test agent was added to 8 mL of Quench neutraliser or Dey-Engley neutraliser (DE-N). Twenty-four-hour cultures of L. plantarum were harvested from de Man, Rogosa and Sharpe Agar (MRSA) and used to prepare a 1×10⁵±5×10⁴ CFUmL⁻¹ suspension in de Man, Rogosa and Sharpe Broth (MRSB). The starting inoculum was confirmed by serial dilution and plating out the resultant suspensions. One millilitre of inoculum was added to each sample of neutralised test agent and incubated at 37±2° C. and 5% CO₂ for 24 hours. Following incubation, the number of viable organisms in each suspension were enumerated. Controls were also run simultaneously to assess neutraliser toxicity to the L. plantarum. One millilitre of inoculum was added to both 9 mL neutralising agent and 9 mL Phosphate Buffered Saline (PBS) and incubated at 37±2° C. and 5% CO₂ for 24 hours. Following incubation, the number of viable organisms in each suspension were enumerated.

Phase 2a: Assessment of the Survival Rate of Lactobacillus plantarum Following 24 Hours Contact with Copper Gluconate and Zinc Gluconate at Stock Concentrations, as Single Agents and in Combination.

Twenty-four-hour cultures of L. plantarum were harvested from MRSA and used to prepare a 1×10⁸±5×10⁷ CFUmL⁻¹ suspension in MRSB. The starting inoculum was confirmed by serial dilution and plating out the resultant suspensions. Two grams of copper gluconate and 1.5 g of zinc gluconate were dissolved in separate 10 mL aliquots of deionised water at 37° C.±2° C. Five millilitres of each solution were then mixed to create a 10 mL copper gluconate and zinc gluconate solution. A 50 μL aliquot of each test agent was added to the wells of a 96-well plate. A 50 μL aliquot of L. plantarum inoculum was then added to the test agent and pipette mixed. The 96-well plates were incubated for 24 hours at 37° C.±2° C. and 5% CO₂. Following incubation, 100 μL of DE-N was added to each well and pipette mixed. A 10 μL aliquot of each neutralised suspension was taken from each well and spot-plated onto MRSA. The plates were incubated for 24 hours at 37° C.±2° C. and 5% CO₂. Following incubation, the presence or absence of growth on the plates was recorded. Testing was performed in triplicate.

Phase 2b: Assessment of the Survival Rate of Lactobacillus plantarum Following 24 Hours Contact with Copper Gluconate and Zinc Gluconate at a Range of Concentrations, as Single Agents and in Combination.

Twenty-four-hour cultures of L. plantarum were harvested from MRSA and used to prepare a 1×10⁸±5×10⁷ CFUmL⁻¹ suspension in MRSB. The starting inoculum was confirmed by serial dilution and plating out the resultant suspensions. Two grams of copper gluconate and 1.5 grams of zinc gluconate were dissolved in separate 10 mL samples of deionised water. Five millilitres of each solution were then mixed to create a 10 mL copper gluconate and zinc gluconate solution. Each test agent was diluted 1:2 a total of eight times. A 50 μL aliquot of each test agent dilution was added to the wells of a 96-well plate. A 50 μL aliquot of L. plantarum inoculum was added to the test agent and pipette mixed. The 96-well plates were incubated for 24 hours at 37° C.±2° C. and 5% CO₂. Following incubation, 100 μL of DE-N was added to each well and pipette mixed. A 10 μL aliquot of each neutralised suspension was taken from each well and spot-plated onto MRSA. The plates were incubated for 24 hours at 37° C.±2° C. and 5% CO₂. Following incubation, the presence or absence of growth on the plates was recorded. Testing was performed in triplicate.

Phase 2c: Assessment of the Survival Rate of Lactobacillus plantarum Following 30 Minutes, 4 Hours and 24 Hours Contact with Copper Gluconate and Zinc Gluconate, as Single Agents and in Combination

Twenty-four-hour cultures of L. plantarum were harvested from MRSA and used to prepare a 1×10⁸±5×10⁷ CFUmL⁻¹ suspension in MRSB. The starting inoculum was confirmed by serial dilution and plating out the resultant suspensions. Three concentrations of each test agent were prepared following the results of Phase 2b. A 50 μL aliquot of each test agent dilution was added to the wells of a 96-well plate. A 50 μL aliquot of the L. plantarum inoculum was added to the test agent and pipette mixed. The 96-well plates were incubated for 30 minutes, 4 hours or 24 hours at 37° C.±2° C. and 5% CO₂. Following incubation, 100 μL of DE-N was added to each well and pipette mixed. A 10 μL aliquot of each neutralised suspension was taken from each well and spot-plated onto MRSA. The plates were incubated for 24 hours at 37° C.±2° C. and 5% CO₂. Following incubation, the presence or absence of growth on the plates was recorded. Testing was performed in triplicate.

Phase 3a: To Assess the Antimicrobial Properties of a Product in Suspension Following the Principles of AATCC Test Method 100 Using a Range of Relevant Microorganisms

Preconditioning of Test Agents

One millilitre aliquots of control and test agents were prepared aseptically in universal containers. A 1 mL aliquot of simulated wound fluid (SWF, 1:1 Fetal Calf Serum and Peptone Diluent (comprised of Peptone 1.0 g, Sodium Chloride 8.5 g and Distilled Water 1.0 L)) was added to each control and test agent. Samples were incubated in a humidified environment at 37±2° C. for three days.

Determination of Antimicrobial Capabilities of Test Agents in Suspension Following the Principles of the AATCC Test Method 100

Microbial suspensions were prepared to 1.0×10⁶±5.0×10⁵ CFUmL⁻¹ in liquid media specific to their growth requirements (for example, Tryptone Soya Broth (TSB), Brain Heart Infusion Broth (BHIB), or Sabouraud Dextrose Broth (SDB)). Inoculums were enumerated by performing 10-fold dilutions and plating out the resultant suspensions onto micro-organism specific agar plates (for example, Tryptone Soya Agar (TSA), Sabouraud Dextrose Agar (SDA) or Brain Heart Infusion Agar (BHIA)). One millilitre of each inoculum was added to universals containing control or test agents that had been preconditioned for three days. Seven millilitres of Dey-Engley Neutraliser (DE-N) was immediately added to the negative control samples (0 hours). The resultant suspensions were then vortexed and serially diluted (1:10) before plating onto micro-organism specific agar plates to quantify viable organisms (e.g. TSA, SDA or BHIA) The test samples were incubated for 24 hours at 37±2° C. Following the 24-hour incubation period, 7 mL of DE-N was added to neutralise the test agents. The test samples were then vortexed, serially diluted and plated onto mico-organism specific agar plates to quantify viable organisms. All testing was performed in triplicate.

Data Analysis

Average Log₁₀CFUsample⁻¹ microbial recoveries were calculated and compared to the negative control to give average Log₁₀CFUsample⁻¹ reductions. Recoveries and reductions are presented as mean±standard deviation (SD) from three independent replicates with duplicate repeats for each independent replicate. The minimum limit of detection for this study was 1 Log. A Student's unpaired t-test (two-tailed) was used to assess statistical differences between the Log₁₀CFUsample⁻¹ recovery data from the negative control and test samples. Data was considered statistically significant when p<0.05.

Phase 4: Assessment of the Antibiofilm Capabilities of a Test Product Against Staphylococcus aureus and Pseudomonas aeruginosa 24 Hour and 72 Hour Pre-Formed Biofilms and Candida albicans 72 Hour Pre-Formed Biofilms

Preparation of the Inoculums and Development of the CDC Reactor Biofilms

Twenty-four-hour cultures of Pseudomonas aeruginosa and Staphylococcus aureus were harvested from a Tryptone Soya Agar (TSA) plate and used to prepare 1×10⁸±5×10⁷ CFUmL⁻¹ suspensions. Each bacterial suspension was further diluted in Tryptone Soya Broth (TSB) to prepare 1×10⁷±5×10⁶ CFUmL⁻¹ suspensions. Forty-eight-hour cultures of Candida albicans were harvested from a Sabouraud Dextrose Agar (SDA) plate and used to prepare 1×10⁷±5×10⁶ CFUmL⁻¹ suspensions. The C. albicans inoculum was further diluted in Sabouraud Dextrose Broth (SDB) to prepare a 1×10⁶±5×10⁵ CFUmL⁻¹ suspension. The working inoculums were confirmed by serial dilution and spread plating. Fetal Bovine Serum (FBS) was added to the C. albicans reactor at a final volume of 10% to aid biofilm attachment. The prepared inoculums were used to inoculate separate sterile CDC reactors containing polycarbonate coupons. The reactors were incubated for either 24 hours or 72 hours at 37±2° C. and 50±5 rpm to encourage biofilm growth.

Treatment of Pre-Formed Biofilms

Following 24 hours or 72 hours incubation, pre-formed biofilms attached to polycarbonate coupons were washed three times in sterile Phosphate Buffered Saline (PBS) in order to remove planktonic organisms. The washed biofilms were transferred to bijous and immersed in 2 mL aliquots of the test agents (Formulation 1 and Formulation 2). Negative and positive controls were tested concurrently. Samples were incubated for 24 hours at 37±2° C. All tests were performed in triplicate.

Recovery and Quantification of Remaining Viable Organisms

Following treatment, biofilms were transferred to 2 mL of DE-N and placed into a sonicating water bath for 5 minutes in order to recover remaining attached organisms. The resultant suspensions were serially diluted and 100 μL samples of each dilution were spread onto either TSA or SDA in duplicate. TSA and SDA plates were incubated for 24 hours at 37±2° C. and 48 hours at 30±2° C. respectively, and total viable counts were determined. The number of colonies were counted and expressed as average Log recoveries (Log₁₀CFUmL⁻¹) and average Log reductions (Log₁₀CFUmL⁻¹). Data was presented as the mean±standard deviation (SD) from three independent replicates with duplicate repeats for each independent replicate.

Statistical Analysis

To assess statistical differences between the test samples and the negative control, recovery Log₁₀CFUmL⁻¹ data was analysed using a Student's unpaired t-test (two-tailed). Data was considered statistically significant when p-value <0.05. The minimum limit of detection for this study was 1 Log.

Results

Phase 1a: Neutraliser Validation

The quantity of viable L. plantarum recovered from test agents following neutralisation was significantly different to the amount recovered from control samples (Table 2). The quantity of viable L. plantarum recovered from test agents following neutralisation with DE-N was not significantly different to the amount recovered from control samples. DE-N was selected for use in the following examples as it was able to effectively neutralise the test agents and was not toxic to L. plantarum.

TABLE 2
Neutraliser validation results using Lactobacillus plantarum.

Results

| Test agent | Neutraliser | Toxicity | Efficacy |
|---|---|---|---|
| Copper gluconate | Quench | No | No |
| Zinc gluconate | Quench | No | No |
| Copper gluconate | Quench | No | No |
| and Zinc gluconate | | | |
| Copper gluconate | DE-N | No | Yes |
| Zinc gluconate | DE-N | No | Yes |
| Copper gluconate | DE-N | No | Yes |
| and Zinc gluconate | | | |

Phase 2a: Assessment of the Survival Rate of Lactobacillus plantarum Following 24 Hours Contact with Copper Gluconate and Zinc Gluconate at Stock Concentrations, as Single Agents and in Combination

Growth of L. plantarum was observed following 24 hours contact time with stock solutions of zinc gluconate. No growth of L. plantarum was observed following 24 hours contact time with stock solutions of copper gluconate or copper gluconate in combination with zinc gluconate (Table 3).

TABLE 3
Growth observed after recovering viable Lactobacillus plantarum
following 24 hours contact with test agents (n = 3).

| | Stock concentration | |
|---|---|---|
| Test agent | (%) | Growth |
| Copper gluconate | 8.167 | No |
| Zinc gluconate | 6.326 | Yes |
| Copper gluconate | 4.084/3.163 | No |
| and Zinc gluconate | | |

Phase 2b: Assessment of the Survival Rate of Lactobacillus plantarum Following 24 Hours Contact with Copper Gluconate and Zinc Gluconate at a Range of Concentrations, as Single Agents and in Combination.

The growth of L. plantarum was assessed following treatment with eight concentrations of copper gluconate and zinc gluconate alone and in combination, over a 24 hour contact period. Growth of L. plantarum was observed following 24 hours contact time with 0.255% copper gluconate solutions (Tables 4 and 5). Growth was observed following 24 hours contact time with zinc gluconate at all concentrations (Table 6). Growth was observed following 24 hours contact time with 0.255% copper gluconate and 0.198% zinc gluconate in combination (Tables 4 and 7).

TABLE 4
Minimum concentration of test agents that allowed growth of viable
Lactobacillus plantarum following 24 hours contact time (n = 3).

| | Minimum concentration at |
|---|---|
| Test agent | which growth was observed (%) |
| Copper gluconate | 0.255 |
| Zinc gluconate | 6.326 |
| Copper gluconate | 0.255/0.198 |
| and zinc gluconate | |

TABLE 5
Growth observed after recovering viable Lactobacillus plantarum
following challenge with eight concentrations of
copper gluconate for 24 hours.

| | Copper gluconate | | |
|---|---|---|---|
| Concentration | N = 1 | N = 2 | N = 3 |

| (%) | 1.1 | 1.2 | 2.1 | 2.2 | 3.1 | 3.2 |
|---|---|---|---|---|---|---|
| 8.167 | − | − | − | − | − | − |
| 4.084 | − | − | − | − | − | − |
| 2.042 | − | − | − | − | − | − |
| 1.021 | − | − | − | − | − | − |
| 0.510 | − | − | − | − | − | − |
| 0.255 | + | + | + | + | + | + |
| 0.128 | + | + | + | + | + | + |
| 0.064 | + | + | + | + | + | + |

+ = growth present,
− = no growth present.

TABLE 6
Growth observed after recovering viable Lactobacillus plantarum
following treatment with eight concentrations of
zinc gluconate for 24 hours.

| | Zinc gluconate | | |
|---|---|---|---|
| Concentration | N = 1 | N = 2 | N = 3 |

| (%) | 1.1 | 1.2 | 2.1 | 2.2 | 3.1 | 3.2 |
|---|---|---|---|---|---|---|
| 6.326 | + | + | + | + | + | + |
| 3.163 | + | + | + | + | + | + |
| 1.582 | + | + | + | + | + | + |
| 0.791 | + | + | + | + | + | + |
| 0.395 | + | + | + | + | + | + |
| 0.198 | + | + | + | + | + | + |
| 0.099 | + | + | + | + | + | + |
| 0.049 | + | + | + | + | + | + |

+ = growth present.

TABLE 7
Growth observed after recovering viable Lactobacillus plantarum
following treatment with eight concentrations of copper
gluconate and zinc gluconate in combination.

| Concentration | Concentration | | | | | | |
|---|---|---|---|---|---|---|---|
| Copper | Zinc | Copper gluconate and Zinc gluconate | | | | | |
| gluconate | gluconate | N = 1 | | N = 2 | | N = 3 | |
| (%) | (%) | 1.1 | 1.2 | 2.1 | 2.2 | 3.1 | 3.2 |
| 4.084 | 3.163 | − | − | − | − | − | − |
| 2.042 | 1.582 | − | − | − | − | − | − |
| 1.021 | 0.791 | − | − | − | − | − | − |
| 0.511 | 0.395 | − | − | − | − | − | − |
| 0.255 | 0.198 | + | + | + | + | + | + |
| 0.128 | 0.099 | + | + | + | + | + | + |
| 0.064 | 0.049 | + | + | + | + | + | + |
| 0.032 | 0.025 | + | + | + | + | + | + |

+ = growth present,
− = no growth present.

Phase 2c: Assessment of the Survival Rate of Lactobacillus plantarum Following 30 Minutes, 4 Hours and 24 Hours Contact with Copper Gluconate and Zinc Gluconate, as Single Agents and in Combination.

As the contact time increased the concentration of copper gluconate required to inhibit growth of L. plantarum decreased. Growth was observed in two of three replicates following 30 minutes contact time with 8.167% copper gluconate, however consistent growth was observed across all replicates following 30 minutes contact time with ≤4.083% copper gluconate (Tables 8 and 9). The minimum concentration for L. plantarum growth was much lower following 4 hour and 24 hour contact times, with growth occurring at 0.510% and 0.255% copper gluconate respectively (Tables 8, 12 and 15).

Growth and survival of L. plantarum was consistently observed following 30 minutes, 4 hours and 24 hours contact time with ≤6.326% zinc gluconate when tested as a single agent (Tables 8, 10, 13 and 16).

Lactobacillus plantarum was unable to survive following 4 hours and 24 hours contact with copper gluconate concentrations of 1.021% and 0.510% respectively (Tables 13 and 15). However, when in combination with zinc gluconate, Lactobacillus plantarum was able to grow following 4 and 24 hours contact time with comparatively higher concentrations of copper gluconate (2.042% and 0.511%, respectively), thereby suggesting zinc gluconate may inhibit the growth-reducing effects of copper gluconate (Tables 14 and 17).

TABLE 8
Minimum concentration of test agents that allowed
for growth of viable Lactobacillus plantarum
following various contact times (n = 3).

| | Minimum concentration at | | |
|---|---|---|---|
| | which growth was observed (%) | | |
| Test agent | 30 minutes | 4 hours | 24 hours |
| Copper gluconate | 8.167 | 0.510 | 0.255 |
| Zinc gluconate | 6.326 | 6.326 | 6.326 |
| Copper gluconate | 4.084/3.163 | 2.042/3.163 | 1.021/3.163 |
| and Zinc gluconate | | | |

TABLE 9
Growth observed after recovering viable Lactobacillus plantarum
following challenge with three concentrations of
copper gluconate for 30 minutes.

| | Copper gluconate | | |
|---|---|---|---|
| Concentration | N = 1 | N = 2 | N = 3 |

| (%) | 1.1 | 1.2 | 2.1 | 2.2 | 3.1 | 3.2 |
|---|---|---|---|---|---|---|
| 8.167 | − | − | − | + | − | + |
| 4.084 | + | + | + | + | + | + |
| 2.042 | + | + | + | + | + | + |

+ = growth present,
− = no growth present.

TABLE 10
Growth observed after recovering viable Lactobacillus plantarum
following challenge with three concentrations of
zinc gluconate for 30 minutes.

| | Zinc gluconate | | |
|---|---|---|---|
| Concentration | N = 1 | N = 2 | N = 3 |

| (%) | 1.1 | 1.2 | 2.1 | 2.2 | 3.1 | 3.2 |
|---|---|---|---|---|---|---|
| 6.326 | + | + | + | + | + | + |
| 3.163 | + | + | + | + | + | + |
| 1.5815 | + | + | + | + | + | + |

+ = growth present,
− = no growth present.

TABLE 11
Growth observed after recovering viable Lactobacillus plantarum
following challenge with three concentrations of copper gluconate
and zinc gluconate in combination for 30 minutes.

| Concentration | Concentration | | | | | | |
|---|---|---|---|---|---|---|---|
| Copper | Zinc | Copper gluconate and Zinc gluconate | | | | | |
| gluconate | gluconate | N = 1 | | N = 2 | | N = 3 | |
| (%) | (%) | 1.1 | 1.2 | 2.1 | 2.2 | 3.1 | 3.2 |
| 4.084 | 3.163 | + | + | + | + | + | + |
| 2.042 | 3.163 | + | + | + | + | + | + |
| 1.021 | 3.163 | + | + | + | + | + | + |

+ = growth present,
− = no growth present.

TABLE 12
Growth observed after recovering viable Lactobacillus plantarum
following challenge with three concentrations
of copper gluconate for 4 hours.

| | Copper gluconate | | |
|---|---|---|---|
| Concentration | N = 1 | N = 2 | N = 3 |

| (%) | 1.1 | 1.2 | 2.1 | 2.2 | 3.1 | 3.2 |
|---|---|---|---|---|---|---|
| 1.021 | − | − | − | − | − | − |
| 0.510 | + | + | + | + | + | + |
| 0.255 | + | + | + | + | + | + |

+ = growth present,
− = no growth present.

TABLE 13
Growth observed after recovering viable Lactobacillus plantarum
following challenge with three concentrations
of zinc gluconate for 4 hours.

| | Zinc gluconate | | |
|---|---|---|---|
| Concentration | N = 1 | N = 2 | N = 3 |

| (%) | 1.1 | 1.2 | 2.1 | 2.2 | 3.1 | 3.2 |
|---|---|---|---|---|---|---|
| 6.326 | + | + | + | + | + | + |
| 3.163 | + | + | + | + | + | + |
| 1.5815 | + | + | + | + | + | + |

+ = growth present,
− = no growth present.

TABLE 14
Growth observed after recovering viable Lactobacillus plantarum
following challenge with three concentrations of copper gluconate
and zinc gluconate in combination for 4 hours.

| Concentration | Concentration | | | | | | |
|---|---|---|---|---|---|---|---|
| Copper | Zinc | Copper gluconate and Zinc gluconate | | | | | |
| gluconate | gluconate | N = 1 | | N = 2 | | N = 3 | |
| (%) | (%) | 1.1 | 1.2 | 2.1 | 2.2 | 3.1 | 3.2 |
| 4.084 | 3.163 | − | − | − | − | − | − |
| 2.042 | 3.163 | + | + | + | + | + | + |
| 1.021 | 3.163 | + | + | + | + | + | + |

+ = growth present,
− = no growth present.

TABLE 15
Growth observed after recovering viable Lactobacillus plantarum
following challenge with three concentrations of
copper gluconate for 24 hours.

| | Copper gluconate | | |
|---|---|---|---|
| Concentration | N = 1 | N = 2 | N = 3 |

| (%) | 1.1 | 1.2 | 2.1 | 2.2 | 3.1 | 3.2 |
|---|---|---|---|---|---|---|
| 0.510 | − | − | − | − | − | − |
| 0.255 | + | + | + | + | + | + |
| 0.128 | + | + | + | + | + | + |

+ = growth present,
− = no growth present.

TABLE 16
Growth observed after recovering viable Lactobacillus plantarum
following challenge with three concentrations of
zinc gluconate for 24 hours.

| | Zinc gluconate | | |
|---|---|---|---|
| Concentration | N = 1 | N = 2 | N = 3 |

| (%) | 1.1 | 1.2 | 2.1 | 2.2 | 3.1 | 3.2 |
|---|---|---|---|---|---|---|
| 6.326 | + | + | + | + | + | + |
| 3.163 | + | + | + | + | + | + |
| 1.5815 | + | + | + | + | + | + |

+ = growth present,
− = no growth present.

TABLE 17
Growth observed after recovering viable Lactobacillus plantarum
following challenge with three concentrations of copper gluconate
and zinc gluconate in combination for 24 hours.

| Concentration | Concentration | | | | | | |
|---|---|---|---|---|---|---|---|
| Copper | Zinc | Copper gluconate and Zinc gluconate | | | | | |
| gluconate | gluconate | N = 1 | | N = 2 | | N = 3 | |
| (%) | (%) | 1.1 | 1.2 | 2.1 | 2.2 | 3.1 | 3.2 |
| 1.021 | 3.163 | − | − | − | + | + | + |
| 0.511 | 3.163 | + | + | + | + | + | + |
| 0.255 | 3.163 | + | + | + | + | + | + |

+ = growth present,
− = no growth present.

### Discussion

Traditionally, probiotics are live, non-pathogenic microorganism (or lysates thereof or tyndallizates thereof) which, when administered in adequate amounts, provide a health benefit to the host. The results show that Lactobacillus plantarum, a Gram-positive bacteria was able to grow following 4 and 24 hours contact time with higher concentration of copper gluconate when in combination with zinc gluconate suggesting zinc gluconate may inhibit the growth-reducing effects of copper gluconate

Phase 3. Determination of Antimicrobial Capabilities of Test Agents in Suspension Following the Principles of the AATCC Test Method 100

Gram Negative Bacteria

Acinetobacter baumannii

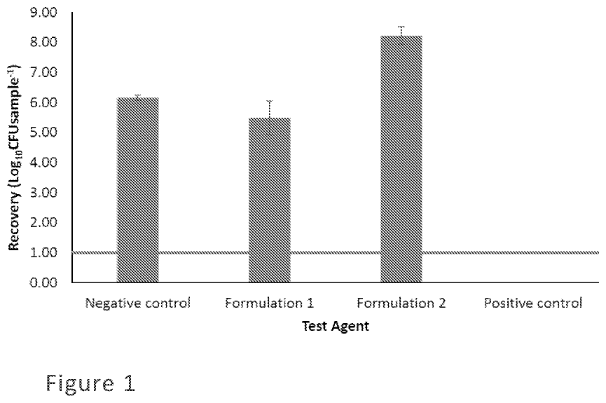

An average of 6.05±0.08 Log₁₀CFUsample⁻¹ Acinetobacter baumannii was recovered from the negative control sample at 0 hours. An average of 5.39±0.56 and 8.12±0.30 Log₁₀CFUsample⁻¹ viable A. baumannii were recovered following 24 hours treatment with Formulations 1 and 2, respectively. Thus 24 hour treatment with Formulation 1 resulted in a 0.66±0.56 Log₁₀CFUsample⁻¹ reduction compared to the negative control at 0 hours (Table 18, FIG. 1).

TABLE 18
Average recovery of Acinetobacter baumannii and average reductions in the quantity
of viable Acinetobacter baumannii recovered per sample from test agents compared
to the negative control at 0 hours. Formulation 1 - 0.2% Copper gluconate and 0.1% zinc
gluconate with Lactobacillus plantarum. Formulation 2 - 0.2% Copper gluconate and
0.1% zinc gluconate without Lactobacillus plantarum.

| | Average | Average | |
|---|---|---|---|
| | ecovery ± SD | reduction ± SD | Statistical |
| Test agent | (Log₁₀CFUsample⁻¹) | (Log₁₀CFUsample⁻¹) | significance |
| Negative control | 6.05 ± 0.08 | N/A | N/A |
| Formulation 1 | 5.39 ± 0.56 | 0.66 ± 0.56 | — |
| Formulation 2 | 8.12 ± 0.30 | No reduction | N/A |
| Positive control | 0.00 ± 0.00 | 6.18 ± 0.00 | *** |

CFU = colony forming units, N/A = not applicable, SD = standard deviation, * = p < 0.05, ** = p < 0.01, *** = p < 0.001.

Campylobacter coli

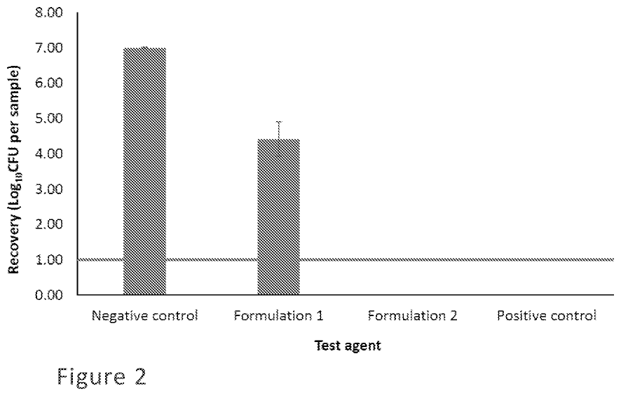

An average of 6.99±0.02 Log₁₀CFUsample⁻¹ Campylobacter coli was recovered from the negative control sample at 0 hours. An average of 4.41±0.48 Log₁₀CFUsample⁻¹ viable C. coli was recovered following a 24 hour treatment with Formulation 1. Following 24 hours incubation no viable C. coli were recovered from the Formulation 2 treated group. So, 24 hour treatment with Formulation 1 and Formulation 2 resulted in a 2.58±0.48 and 6.99±0.00 Log₁₀CFUsample⁻¹ reduction compared to the negative control at 0 hours, respectively (Table 19, FIG. 2).

TABLE 19
Average recovery of Campylobacter coli and average reductions in the quantity
of viable Campylobacter coli recovered per sample from test agents compared
to the negative control at 0 hours. Formulation 1 - 0.2% Copper gluconate and 0.1%
zinc gluconate with Lactobacillus plantarum. Formulation 2 - 0.2% Copper gluconate
and 0.1% zinc gluconate without Lactobacillus plantarum.

| | Average | Average | |
|---|---|---|---|
| | recovery ± SD | reduction ± SD | Statistical |
| Test agent | (Log₁₀CFUsample⁻¹) | (Log₁₀CFUsample⁻¹) | significance |
| Negative control | 6.99 ± 0.02 | N/A | N/A |
| Formulation 1 | 4.41 ± 0.48 | 2.58 ± 0.48 | *** |
| Formulation 2 | 0.00 ± 0.00 | 6.99 ± 0.00 | *** |
| Positive control | 0.00 ± 0.00 | 6.99 ± 0.00 | *** |

CFU = colony forming units, N/A = not applicable, SD = standard deviation, * = p < 0.05, ** = p < 0.01, *** = p < 0.001.

Enterobacter cloacae

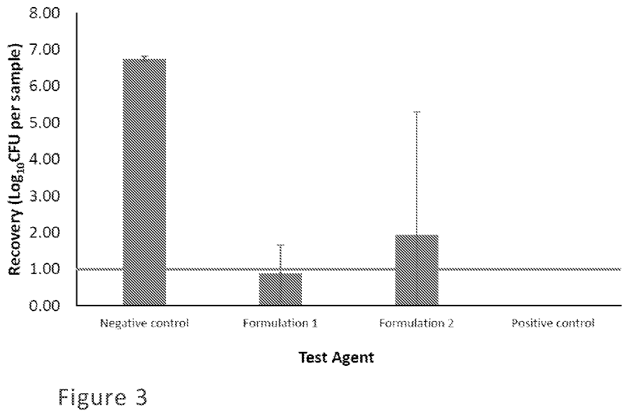

An average of 6.74±0.06 Log₁₀CFUsample⁻¹ Enterobacter cloacae was recovered from the negative control sample at 0 hours. An average of 0.88±0.78 and 1.94±3.36 Log₁₀CFUsample⁻¹ viable E. cloacae were recovered following 24 hours treatment with Formulations 1 and 2, respectively. These were reductions of 5.86±0.78 Log₁₀CFUsample⁻¹ and no reduction respectively, compared to the negative control at 0 hours (Table 20, FIG. 3).

TABLE 20
Average recovery of Enterobacter cloacae and average reductions in the quantity
of viable Enterobacter cloacae recovered per sample from test agents compared
to the negative control at 0 hours. Formulation 1 - 0.2% Copper gluconate and 0.1%
zinc gluconate with Lactobacillus plantarum. Formulation 2 - 0.2% Copper gluconate
and 0.1% zinc gluconate without Lactobacillus plantarum.

| | Average | Average | |
|---|---|---|---|
| | recovery ± SD | reduction ± SD | Statistical |
| Test agent | (Log₁₀CFUsample⁻¹) | (Log₁₀CFUsample⁻¹) | significance |
| Negative control | 6.74 ± 0.06 | N/A | N/A |
| Formulation 1 | 0.88 ± 0.78 | 5.86 ± 0.78 | *** |
| Formulation 2 | 1.94 ± 3.36 | No reduction | — |
| Positive control | 0.00 ± 0.00 | 6.74 ± 0.00 | *** |

CFU = colony forming units, N/A = not applicable, SD = standard deviation, * = p < 0.05, ** = p < 0.01, *** = p < 0.001.

Haemophilus influenzae

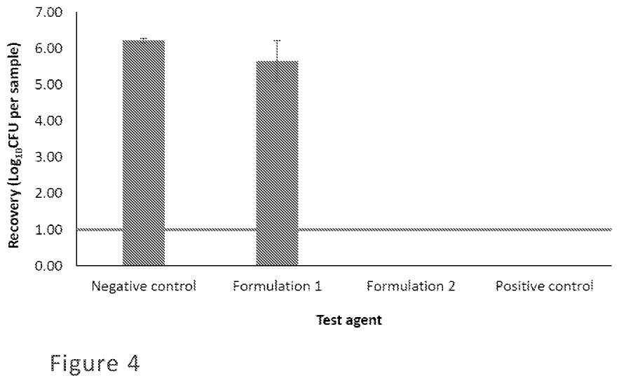

An average of 6.22±0.06 Log₁₀CFUsample⁻¹ Haemophilus influenzae was recovered from the negative control sample at 0 hours. An average of 5.66±0.56 Log₁₀CFUsample⁻¹ viable H. influenzae was recovered following a 24 hour treatment with Formulation 1. Following 24 hours incubation, no viable H. influenzae were recovered from the Formulation 2 treated sample. These were reductions of 0.57±0.56 and 6.22±0.00 Log₁₀CFUsample⁻¹ respectively, compared to the negative control at 0 hours (Table 21, FIG. 4).

TABLE 21
Average recovery of Haemophilus influenzae and average reductions in the quantity
of viable Haemophilus influenzae recovered per sample from test agents compared
to the negative control at 0 hours. Formulation 1 - 0.2% Copper gluconate and 0.1% zinc
gluconate with Lactobacillus plantarum. Formulation 2 - 0.2% Copper gluconate
and 0.1% zinc gluconate without Lactobacillus plantarum.

| | Average | Average | |
|---|---|---|---|
| | recovery ± SD | reduction ± SD | Statistical |
| Test agent | (Log₁₀CFUsample⁻¹) | (Log₁₀CFUsample⁻¹) | significance |
| Negative control | 6.22 ± 0.06 | N/A | N/A |
| Formulation 1 | 5.66 ± 0.56 | 0.57 ± 0.56 | — |
| Formulation 2 | 0.00 ± 0.00 | 6.22 ± 0.00 | *** |
| Positive control | 0.00 ± 0.00 | 6.22 ± 0.00 | *** |

CFU = colony forming units, N/A = not applicable, SD = standard deviation, * = p < 0.05, ** = p < 0.01, *** = p < 0.001.

Helicobacter pylori

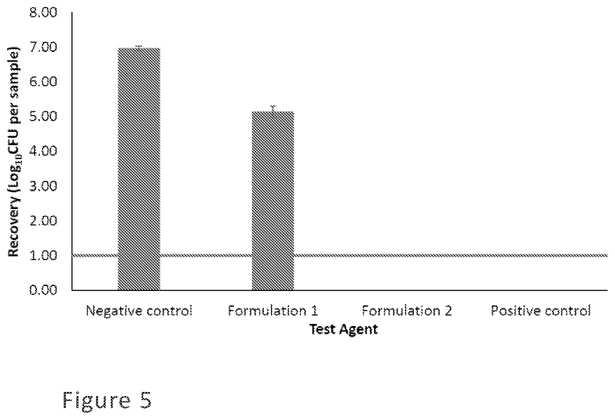

An average of 6.97±0.06 Log₁₀CFUsample⁻¹ Helicobacter pylori was recovered from the negative control sample at 0 hours. An average of 5.15±0.17 Log₁₀CFUsample⁻¹ viable Helicobacter pylori was recovered following a 24 hour treatment with Formulation 1. No viable H. pylori were recovered from the formulation 2 treated sample, following 24 hours incubation. These were reductions of 1.82±0.17 and 6.97±0.00 Log₁₀CFUsample⁻¹ respectively, compared to the negative control at 0 hours (Table 22, FIG. 5).

TABLE 22
Average recovery of Helicobacter pylori and average reductions in the quantity
of viable Helicobacter pylori recovered per sample from test agents compared
to the negative control at 0 hours. Formulation 1 - 0.2% Copper gluconate and 0.1%
zinc gluconate with Lactobacillus plantarum. Formulation 2 - 0.2% Copper gluconate
and 0.1% zinc gluconate without Lactobacillus plantarum.

| | Average | Average | |
|---|---|---|---|
| | recovery ± SD | reduction ± SD | Statistical |
| Test agent | (Log₁₀CFUsample⁻¹) | (Log₁₀CFUsample⁻¹) | significance |
| Negative control | 6.97 ± 0.06 | N/A | N/A |
| Formulation 1 | 5.15 ± 0.17 | 1.82 ± 0.17 | *** |
| Formulation 2 | 0.00 ± 0.00 | 6.97 ± 0.00 | *** |
| Positive control | 0.00 ± 0.00 | 6.97 ± 0.00 | *** |

CFU = colony forming units, N/A = not applicable, SD = standard deviation, * = p < 0.05, ** = p < 0.01, *** = p < 0.001.

Pseudomonas aeruginosa (Phase 3 Data)

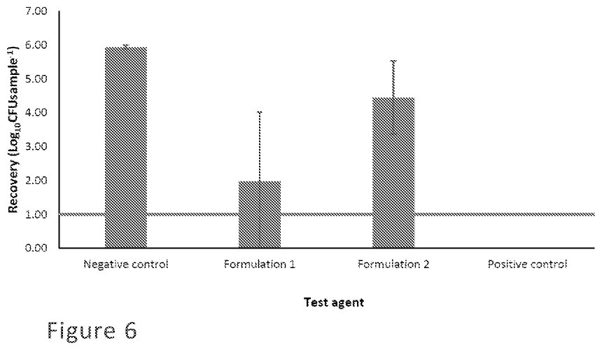

An average of 5.94±0.06 Log₁₀CFUsample⁻¹ Pseudomonas aeruginosa was recovered from the negative control sample at 0 hours. An average of 1.99±2.57 and 4.46±1.08 Log₁₀CFUsample⁻¹ viable P. aeruginosa were recovered following 24 hours treatment with Formulations 1 and 2 respectively (Table 19, FIG. 2). Therefore, twenty-four hour treatment with Formulation 1 resulted in a reduction of 3.96±2.57 Log₁₀CFUsample⁻¹ compared to the negative control (p<0.05). Whereas twenty-four hour treatment with Formulation 2 resulted in a reduction of 1.48±1.08 Log₁₀CFUsample⁻¹ compared to the negative control (Table 23, FIG. 6).

TABLE 23
Average recovery of Pseudomonas aeruginosa and average reductions in the quantity
of viable Pseudomonas aeruginosa recovered per sample from test agents compared
to the negative control at 0 hours. Formulation 1 - 0.2% Copper gluconate and 0.1% zinc
gluconate with Lactobacillus plantarum. Formulation 2 - 0.2% Copper gluconate
and 0.1% zinc gluconate without Lactobacillus plantarum.

| | Average | Average | |
|---|---|---|---|
| | recovery ± SD | reduction ± SD | Statistical |
| Test agent | (Log₁₀CFUsample⁻¹) | (Log₁₀CFUsample⁻¹) | significance |
| Negative control | 5.94 ± 0.06 | N/A | N/A |
| Formulation 1 | 1.99 ± 2.57 | 3.96 ± 2.57 | * |
| Formulation 2 | 4.46 ± 1.08 | 1.48 ± 1.08 | — |
| Positive control | 0.00 ± 0.00 | 5.94 ± 0.00 | *** |

CFU = colony forming units, N/A = not applicable, SD = standard deviation, — = p > 0.05, * = p < 0.05, *** = p < 0.001.

Pseudomonas aeruginosa (Phase 5 Data)

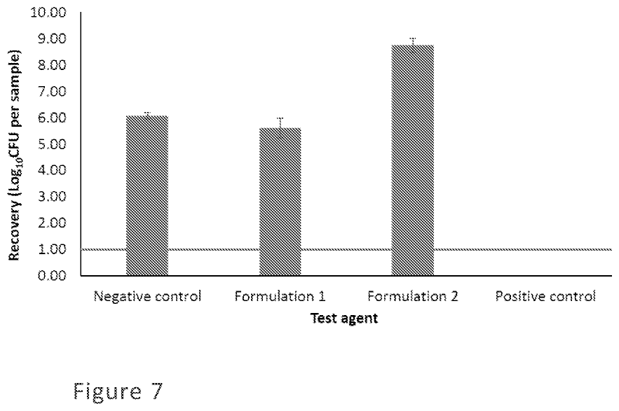

An average of 5.98±0.12 Log₁₀CFUsample⁻¹ Pseudomonas aeruginosa was recovered from the negative control sample at 0 hours. An average of 5.51±0.38 and 8.65±0.26 Log₁₀CFUsample⁻¹ viable P. aeruginosa were recovered following 24 hours treatment with Formulations 1 and 2, respectively. There were no significant Log reductions following treatment with Formulation 1 or Formulation 2, compared to the negative control at 0 hours (Table 24, FIG. 7).

TABLE 24
Average recovery of Pseudomonas aeruginosa and average reductions in the quantity
of viable Pseudomonas aeruginosa recovered per sample from test agents compared
to the negative control at 0 hours. Formulation 1-0.2% Copper gluconate and 0.1% zinc
gluconate with Lactobacillus plantarum. Formulation 2-0.2% Copper gluconate and
0.1% zinc gluconate without Lactobacillus plantarum.

| | Average recovery ± SD | Average reduction ± SD | Statistical |
|---|---|---|---|
| Test agent | (Log₁₀CFUsample⁻¹) | (Log₁₀CFUsample⁻¹) | significance |
| Negative control | 5.98 ± 0.12 | N/A | N/A |
| Formulation 1 | 5.51 ± 0.38 | 0.47 ± 0.38 | — |
| Formulation 2 | 8.65 ± 0.26 | No reduction | N/A |
| Positive control | 0.00 ± 0.00 | 5.98 ± 0.00 | *** |

CFU = colony forming units,
N/A = not applicable,
SD = standard deviation,
* = p < 0.05,
** = p < 0.01,
*** = p < 0.001.

Salmonella enteritidis

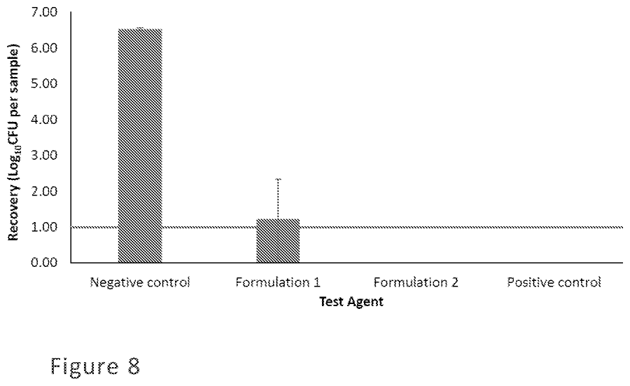

An average of 6.53±0.04 Log₁₀CFUsample⁻¹ Salmonella enteritidis was recovered from the negative control sample at 0 hours. An average of 1.22±1.12 Log₁₀CFUsample⁻¹ viable S. enteritidis was recovered following a 24 hour treatment with Formulation 1. No viable S. enteritidis were recovered from the Formulation 2 treated sample, following 24 hours incubation. These were reductions of 5.31±1.12 and 6.53±0.00 Log₁₀CFUsample⁻¹ respectively, compared to the negative control at 0 hours (Table 25, FIG. 8).

TABLE 25
Average recovery of Salmonella enteritidis and average reductions in the quantity
of viable Salmonella enteritidis recovered per sample from test agents compared
to the negative control at 0 hours. Formulation 1-0.2% Copper gluconate and 0.1% zinc
gluconate with Lactobacillus plantarum. Formulation 2-0.2% Copper gluconate and
0.1% zinc gluconate without Lactobacillus plantarum.

| | Average recovery ± SD | Average reduction ± SD | Statistical |
|---|---|---|---|
| Test agent | (Log₁₀CFUsample⁻¹) | (Log₁₀CFUsample⁻¹) | significance |
| Negative control | 6.53 ± 0.04 | N/A | N/A |
| Formulation 1 | 1.22 ± 1.12 | 5.31 ± 1.12 | *** |
| Formulation 2 | 0.00 ± 0.00 | 6.53 ± 0.00 | *** |
| Positive control | 0.00 ± 0.00 | 6.53 ± 0.00 | *** |

CFU = colony forming units,
N/A = not applicable,
SD = standard deviation,
* = p < 0.05,
** = p < 0.01,
*** = p < 0.001.

Gram Positive Bacteria

Enterococcus faecium

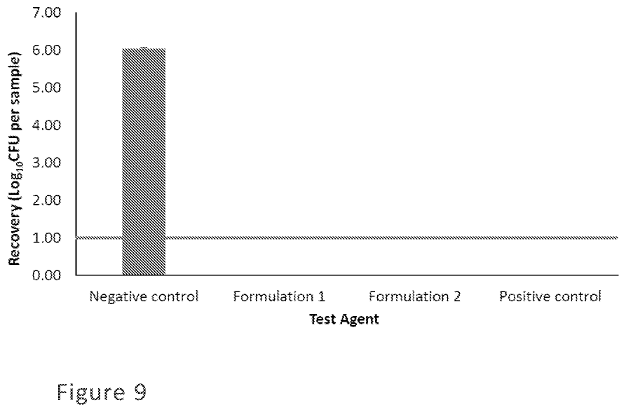

An average of 6.03±0.05 Log₁₀CFUsample⁻¹ Enterococcus faecium was recovered from the negative control sample at 0 hours. Following 24 hours incubation no viable E. faecium were recovered from the samples treated with Formulation 1 or 2. These were reductions of 6.03±0.05 and 6.03±0.05 Log₁₀CFUsample⁻¹ respectively, compared to the negative control at 0 hours (Table 26, FIG. 9).

TABLE 26
Average recovery of Enterococcus faecium and average reductions in the quantity
of viable Enterococcus faecium recovered per sample from test agents compared
to the negative control at 0 hours. Formulation 1-0.2% Copper gluconate and 0.1% zinc
gluconate with Lactobacillus plantarum. Formulation 2-0.2% Copper gluconate and
0.1% zinc gluconate without Lactobacillus plantarum.

| | Average recovery ± SD | Average reduction ± SD | Statistical |
|---|---|---|---|
| Test agent | (Log₁₀CFUsample⁻¹) | (Log₁₀CFUsample⁻¹) | significance |
| Negative control | 6.03 ± 0.05 | N/A | N/A |
| Formulation 1 | 0.00 ± 0.00 | 6.03 ± 0.05 | *** |
| Formulation 2 | 0.00 ± 0.00 | 6.03 ± 0.05 | *** |
| Positive control | 0.00 ± 0.00 | 6.03 ± 0.05 | *** |

CFU = colony forming units,
N/A = not applicable,
SD = standard deviation,
* = p < 0.05,
** = p < 0.01,
*** = p < 0.001.

Neisseria gonorrhoeae

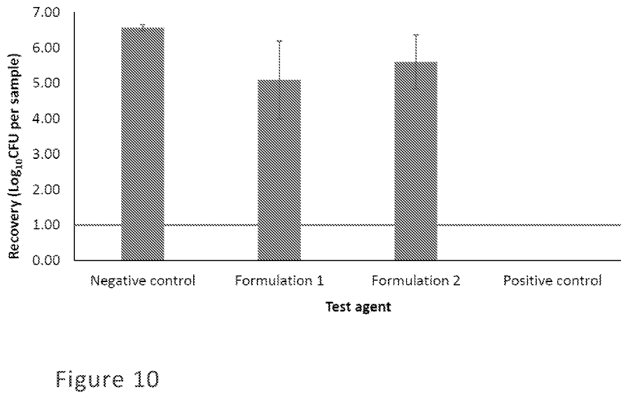

An average of 6.57±0.08 Log₁₀CFUsample⁻¹ Neisseria gonorrhoeae was recovered from the negative control sample at 0 hours. An average of 5.10±1.09 and 5.61±0.76 Log₁₀CFUsample⁻¹ viable Neisseria gonorrhoeae were recovered following 24 hours treatment with Formulations 1 and 2, respectively. These were reductions of 0.43±0.03 and 0.79±0.10 Log₁₀CFUsample⁻¹ respectively, compared to the negative control at 0 hours (Table 27, FIG. 10).

TABLE 27
Average recovery of Neisseria gonorrhoeae and average reductions in the quantity
of viable Neisseria gonorrhoeae recovered per sample from test agents compared
to the negative control at 0 hours. Formulation 1-0.2% Copper gluconate and 0.1% zinc
gluconate with Lactobacillus plantarum. Formulation 2-0.2% Copper gluconate and
0.1% zinc gluconate without Lactobacillus plantarum.

| | Average recovery ± SD | Average reduction ± SD | Statistical |
|---|---|---|---|
| Test agent | (Log₁₀CFUsample⁻¹) | (Log₁₀CFUsample⁻¹) | significance |
| Negative control | 6.57 ± 0.08 | N/A | N/A |
| Formulation 1 | 5.10 ± 1.09 | 0.43 ± 0.03 | — |
| Formulation 2 | 5.61 ± 0.76 | 0.79 ± 0.10 | — |
| Positive control | 0.00 ± 0.00 | 6.57 ± 0.00 | *** |

CFU = colony forming units,
N/A = not applicable,
SD = standard deviation,
* = p < 0.05,
** = p < 0.01,
*** = p < 0.001.

Staphylococcus aureus (Phase 3 Data)

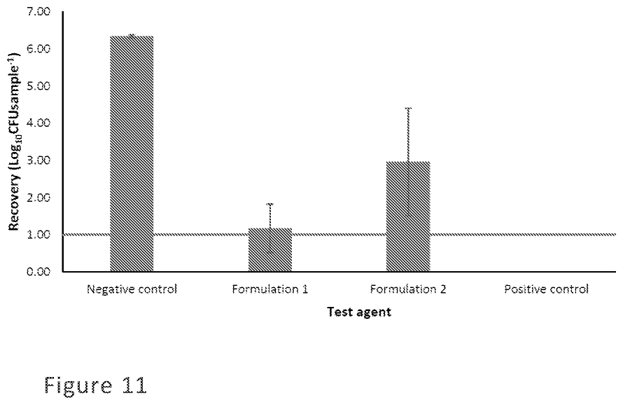

An average of 6.35±0.03 Log₁₀CFUsample⁻¹ Staphylococcus aureus was recovered from the negative control sample at 0 hours. An average of 1.17±1.23 and 2.96±1.75 Log₁₀CFUsample⁻¹ viable S. aureus were recovered following 24 hours treatment with Formulations 1 and 2, respectively. These were reductions of 5.17±1.23 and 3.39±1.75 Log₁₀CFUsample⁻¹, respectively, compared to the negative control at 0 hours (p<0.01 and p<0.05, respectively) (Table 28, FIG. 11).

TABLE 28
Average recovery of Staphylococcus aureus and average reductions in the quantity
of viable Staphylococcus aureus per sample recovered from test agents compared
to the negative control at 0 hours. Formulation 1-0.2% Copper gluconate and 0.1% zinc
gluconate with Lactobacillus plantarum. Formulation 2-0.2% Copper gluconate and
0.1% zinc gluconate without Lactobacillus plantarum.

| | Average recovery ± SD | Average reduction ± SD | Statistical |
|---|---|---|---|
| Test agent | (Log₁₀CFUsample⁻¹) | (Log₁₀CFUsample⁻¹) | significance |
| Negative control | 6.35 ± 0.03 | N/A | N/A |
| Formulation 1 | 1.17 ± 1.23 | 5.17 ± 1.23 | ** |
| Formulation 2 | 2.96 ± 1.75 | 3.39 ± 1.75 | * |
| Positive control | 0.00 ± 0.00 | 6.35 ± 0.00 | *** |

CFU = colony forming units,
N/A = not applicable,
SD = standard deviation,
* = p < 0.05,
** = p < 0.01,
*** = p < 0.001.

Staphylococcus aureus (Phase 5 Data)

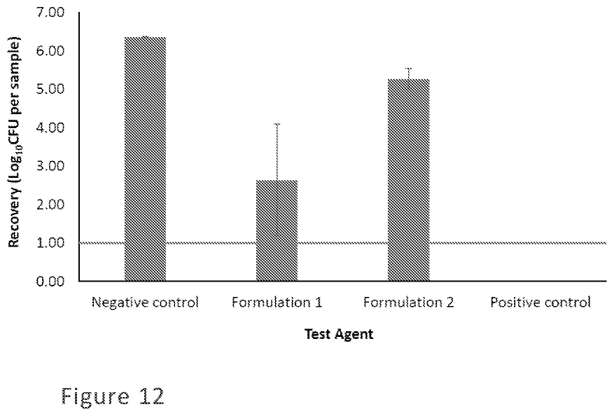

An average of 6.36±0.00 Log₁₀CFUsample⁻¹ Staphylococcus aureus was recovered from the negative control sample at 0 hours. An average of 2.64±1.45 and 5.27±0.26 Log₁₀CFUsample⁻¹ viable Staphylococcus aureus were recovered following 24 hours treatment with Formulations 1 and 2, respectively. These were reductions of 3.72±1.45 and 1.09±0.26 Log₁₀CFUsample⁻¹ respectively, compared to the negative control at 0 hours (Table 29, FIG. 12).

TABLE 29
Average recovery of Staphylococcus aureus and average reductions in the quantity
of viable Staphylococcus aureus recovered per sample from test agents compared
to the negative control at 0 hours. Formulation 1-0.2% Copper gluconate and 0.1% zinc
gluconate with Lactobacillus plantarum. Formulation 2-0.2% Copper gluconate and
0.1% zinc gluconate without Lactobacillus plantarum.

| | Average recovery ± SD | Average reduction ± SD | Statistical |
|---|---|---|---|
| Test agent | (Log₁₀CFUsample⁻¹) | (Log₁₀CFUsample⁻¹) | significance |
| Negative control | 6.36 ± 0.00 | N/A | N/A |
| Formulation 1 | 2.64 ± 1.45 | 3.72 ± 1.45 | * |
| Formulation 2 | 5.27 ± 0.26 | 1.09 ± 0.26 | ** |
| Positive control | 0.00 ± 0.00 | 6.36 ± 0.00 | *** |

CFU = colony forming units,
N/A = not applicable,
SD = standard deviation,
* = p < 0.05,
** = p < 0.01,
*** = p < 0.001.

Streptococcus pneumoniae

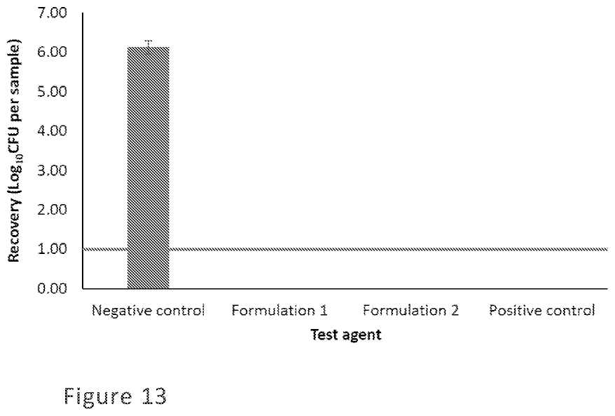

An average of 6.13±0.17 Log₁₀CFUsample⁻¹ Streptococcus pneumoniae was recovered from the negative control sample at 0 hours. Following 24 hours incubation no viable Streptococcus pneumoniae were recovered from samples treated with Formulation 1 or 2. These were reductions of 6.13±0.00 and 6.13±0.00 Log₁₀CFUsample⁻¹ respectively, compared to the negative control at 0 hours (Table 30, FIG. 13).

TABLE 30
Average recovery of Streptococcus pneumoniae and average reductions in the quantity
of viable Streptococcus pneumoniae recovered per sample from test agents compared
to the negative control at 0 hours. Formulation 1-0.2% Copper gluconate and 0.1% zinc
gluconate with Lactobacillus plantarum. Formulation 2-0.2% Copper gluconate and
0.1% zinc gluconate without Lactobacillus plantarum.

| | Average recovery ± SD | Average reduction ± SD | Statistical |
|---|---|---|---|
| Test agent | (Log₁₀CFUsample⁻¹) | (Log₁₀CFUsample⁻¹) | significance |
| Negative control | 6.13 ± 0.17 | N/A | N/A |
| Formulation 1 | 0.00 ± 0.00 | 6.13 ± 0.17 | *** |
| Formulation 2 | 0.00 ± 0.00 | 6.13 ± 0.17 | *** |
| Positive control | 0.00 ± 0.00 | 6.13 ± 0.17 | *** |

CFU = colony forming units,
N/A = not applicable,
SD = standard deviation,
* = p < 0.05,
** = p < 0.01,
*** = p < 0.001.

Fungi

Candida albicans

An average of 6.15±0.11 Log₁₀CFUsample⁻¹ Candida albicans was recovered from the negative control sample at 0 hours. An average of 7.42±0.07 and 5.03±0.05 Log₁₀CFUsample⁻¹ viable C. albicans were recovered following 24 hours treatment with Formulations 1 and 2, respectively (Table 20, FIG. 3).

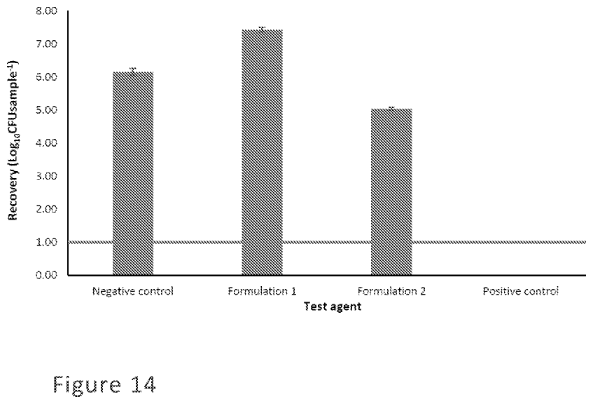

Therefore, twenty-four hour treatment with Formulation 2 resulted in a viable C. albicans reduction of 1.12±0.05 Log₁₀CFUsample⁻¹ compared to the negative control (p<0.001) (Table 31, FIG. 14).

TABLE 31
Average recovery of Candida albicans and average reductions in the quantity
of viable Candida albicans recovered per sample from test agents compared
to the negative control at 0 hours. Formulation 1-0.2% Copper gluconate and 0.1%
zinc gluconate with Lactobacillus plantarum. Formulation 2-0.2% Copper gluconate
and 0.1% zinc gluconate without Lactobacillus plantarum.

| | Average recovery ± SD | Average reduction ± SD | Statistical |
|---|---|---|---|
| Test agent | (Log₁₀CFUsample⁻¹) | (Log₁₀CFUsample⁻¹) | significance |
| Negative control | 6.15 ± 0.11 | N/A | N/A |
| Formulation 1 | 7.42 ± 0.07 | No reduction | N/A |
| Formulation 2 | 5.03 ± 0.05 | 1.12 ± 0.05 | *** |
| Positive control | 0.00 ± 0.00 | 6.15 ± 0.00 | *** |

CFU = colony forming units,
N/A = not applicable,
SD = standard deviation,
*** = p < 0.001.

Candida auris

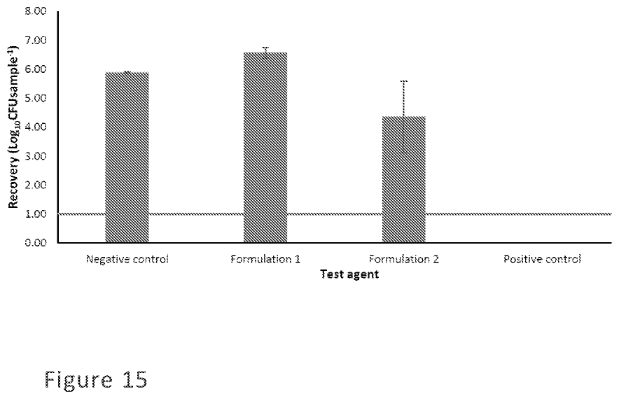

An average of 5.89±0.03 Log₁₀CFUsample⁻¹ Candida auris was recovered from the negative control sample at 0 hours. An average of 6.57±0.17 and 4.36±1.23 Log₁₀CFUsample⁻¹ viable C. auris were recovered following 24 hours treatment with Formulations 1 and 2, respectively. So, twenty-four hour treatment with Formulation 2 resulted in a reduction of 1.53±1.23 Log₁₀CFUsample⁻¹, when compared to the negative control (p<0.05) (Table 32, FIG. 15).

TABLE 32
Average recovery of Candida auris and average reductions in the quantity
of viable Candida auris recovered per sample from test agents compared
to the negative control at 0 hours. Formulation 1-0.2% Copper gluconate and 0.1%
zinc gluconate with Lactobacillus plantarum. Formulation 2-0.2% Copper gluconate
and 0.1% zinc gluconate without Lactobacillus plantarum.

| | Average recovery ± SD | Average reduction ± SD | Statistical |
|---|---|---|---|
| Test agent | (Log₁₀CFUsample⁻¹) | (Log₁₀CFUsample⁻¹) | significance |
| Negative control | 5.89 ± 0.03 | N/A | N/A |
| Formulation 1 | 6.57 ± 0.17 | No reduction | N/A |
| Formulation 2 | 4.36 ± 1.23 | 1.53 ± 1.23 | * |
| Positive control | 0.00 ± 0.00 | 5.89 ± 0.00 | *** |

CFU = colony forming units,
N/A = not applicable,
SD = standard deviation,
* = p < 0.05,
*** = p < 0.001.

Aspergillus brasiliensis

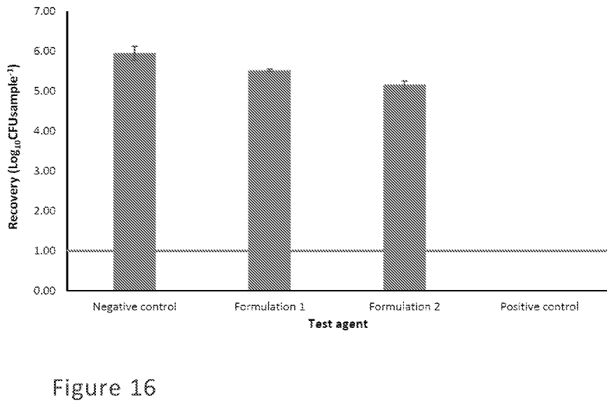

An average of 5.95±0.17 Log₁₀CFUsample⁻¹ Aspergillus brasiliensis was recovered from the negative control sample at 0 hours. An average of 5.52±0.03 and 5.17±0.10 Log₁₀CFUsample⁻¹ viable A. brasiliensis were recovered following 24 hours treatment with Formulations 1 and 2, respectively. These were reductions of 0.43±0.03 and 0.79±0.10 Log₁₀CFUsample⁻¹, respectively, compared to the negative control at 0 hours (p<0.05 and p<0.01, respectively) (Table 33, FIG. 16).

TABLE 33
Average recovery of Aspergillus brasiliensis and average reductions in the quantity
of viable Aspergillus brasiliensis recovered per sample from test agents compared
to the negative control at 0 hours. Formulation 1-0.2% Copper gluconate and 0.1% zinc
gluconate with Lactobacillus plantarum. Formulation 2-0.2% Copper gluconate and
0.1% zinc gluconate without Lactobacillus plantarum.

| | Average recovery ± SD | Average reduction ± SD | Statistical |
|---|---|---|---|
| Test agent | (Log₁₀CFUsample⁻¹) | (Log₁₀CFUsample⁻¹) | significance |
| Negative control | 5.95 ± 0.17 | N/A | N/A |
| Formulation 1 | 5.52 ± 0.03 | 0.43 ± 0.03 | * |
| Formulation 2 | 5.17 ± 0.10 | 0.79 ± 0.10 | ** |
| Positive control | 0.00 ± 0.00 | 5.95 ± 0.00 | *** |

CFU = colony forming units,
N/A = not applicable,
SD = standard deviation,
* = p < 0.05,
** = p < 0.01,
*** = p < 0.001.

### Discussion

The presence of pathogenic microorganisms in a wound can prevent wound healing and lead to chronic infections, which can increase treatment times and costs. Wound treatment products that contain anti-microbial compounds can reduce the microbial load present in the wound and may help to transition the wound to a healing state.

The modified AATCC Test Method is a widely accepted method of testing antimicrobial dressings. The test has been adapted for the treatment of Formulations 1 and 2 in a liquid format. The key principles of the test method include the preconditioning of test agents to provide a greater challenge and comparison of test agent recoveries to a negative control sample recovered at 0 hours.

A greater than 4 Log reduction was achieved for four of the microorganisms tested for both Formulation 1 and Formulation 2. Formulation 1, with the addition of Lactobacillus plantarum, achieved a greater than 4 Log reduction against E. cloacae, E. faecium, S. enteritidis and S. pneumoniae (see FIGS. 3, 9, 8, and 13). Formulation 2, without the addition of L. plantarum, achieved a greater than 4 Log reduction against C. coli, E. faecium, H. influenzae, H. pylori, S. enteritidis and S. pneumoniae (see FIGS. 2, 9, 4, 5, 8 and 13).

The addition of L. plantarum to a combination of 0.2% copper gluconate and 0.1% zinc gluconate significantly increased the antibacterial effect against S. aureus (>3 Log reduction) and E. cloacae (>5 Log reduction) after a 24 hour treatment. The addition of L. plantarum to a combination of 0.2% copper gluconate and 0.1% zinc gluconate significantly decreased the antibacterial effect against C. coli, H. influenzae and H. pylori.

Lactobacillus plantarum naturally produces organic acids, such as lactic acid, acetic acid and propionic acid. The presence of these organic acids lowers the pH of the surrounding environment which has been shown to inhibit the growth of several microorganisms. Haemophilus influenzae and H. pylori are neutralophiles which neutralise acids via production of urease to improve survivability in acidic environments. Campylobacter spp. are sensitive to acidic environments however some strains, such as C. coli, have been found to have an increased tolerance to acidic environments.

The addition of Lactobacillus plantarum to a combination of 0.2% copper gluconate and 0.1% zinc gluconate significantly increased the antimicrobial effect against S. aureus (>4 Log reduction) and P. aeruginosa (>3 Log reduction) after a 24 hour treatment (see FIGS. 6 and 11).

Against C. albicans, C. auris and A. brasiliensis the addition of L. plantarum to copper and zinc gluconate decreased the antimicrobial effect (see FIGS. 14, 15 and 16).

Phase 4: Assessment of the Antibiofilm Capabilities of a Test Product Against Staphylococcus aureus and Pseudomonas Aeruginosa 24 Hour and 72 Hour Pre-Formed Biofilms and Candida albicans 72 Hour Pre-Formed Biofilms

24 Hour Biofilms

Staphylococcus aureus

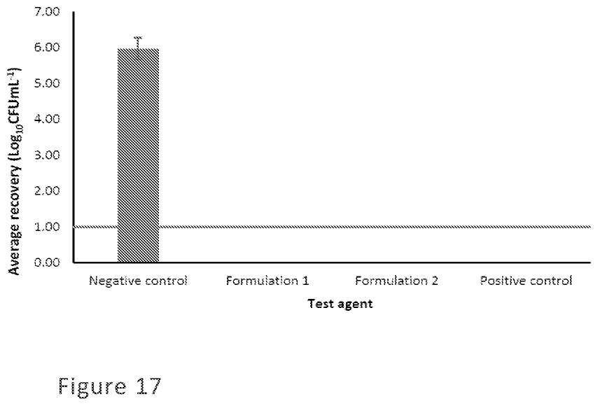

An average of 5.96±0.30 Log₁₀CFUsample⁻¹ viable S. aureus were recovered from the negative control (Table 34, FIG. 17). No viable Staphylococcus aureus was recovered following treatment for 24 hours with Formulation 1 or Formulation 2.

TABLE 34
Average quantities of viable Staphylococcus aureus
recovered and average reductions following treatment for 24 hours
with Formulation 1-0.2% copper gluconate and 0.1% zinc gluconate
with Lactobacillus plantarum and Formulation 2-0.2%
copper gluconate and 0.1% zinc gluconate, when compared to
the negative control.

| | Average recovery ± SD | Average reduction ± SD |
|---|---|---|
| Test agent | (Log₁₀CFUsample⁻¹) | (Log₁₀CFUsample⁻¹) |
| Negative control | 5.96 ± 0.30 | N/A |
| Formulation 1 | 0.00 ± 0.00 | 5.96 ± 0.00* |
| Formulation 2 | 0.00 ± 0.00 | 5.96 ± 0.00* |
| Positive control | 0.00 ± 0.00 | 5.96 ± 0.00* |

SD = standard deviation,
N/A = not applicable,
CFU = colony forming units,
*= p < 0.001.

Pseudomonas aeruginosa

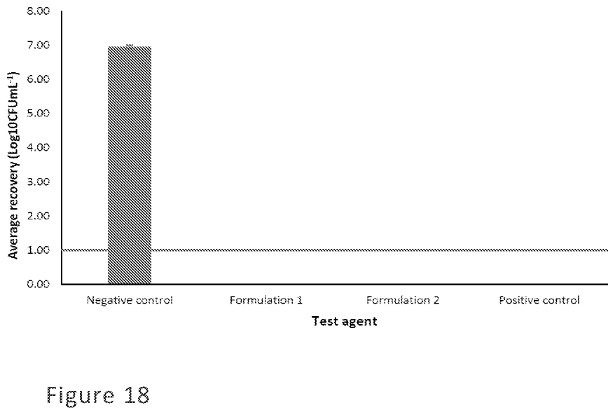

An average of 6.95±0.05 Log₁₀CFUsample⁻¹ viable P. aeruginosa were recovered from the negative control (Table 35, FIG. 18). No viable P. aeruginosa was recovered following treatment for 24 hours with Formulation 1 or Formulation 2.

TABLE 35
Average quantities of viable Pseudomonas aeruginosa
recovered and average reductions following treatment for 24 hours
with Formulation 1-0.2% copper gluconate and0.1% zinc gluconate
with Lactobacillus plantarum and Formulation 2-0.2%
copper gluconate and 0.1% zinc gluconate, when compared to
the negative control.

| | Average recovery ± SD | Average reduction ± SD |
|---|---|---|
| Test agent | (Log₁₀CFUsample⁻¹) | (Log₁₀CFUsample⁻¹) |
| Negative control | 6.95 ± 0.05 | N/A |
| Formulation 1 | 0.00 ± 0.00 | 6.95 ± 0.00* |
| Formulation 2 | 0.00 ± 0.00 | 6.95 ± 0.00* |
| Positive control | 0.00 ± 0.00 | 6.95 ± 0.00* |

SD = standard deviation,
N/A = not applicable,
*= p < 0.001.

72 Hour Biofilms

Staphylococcus aureus

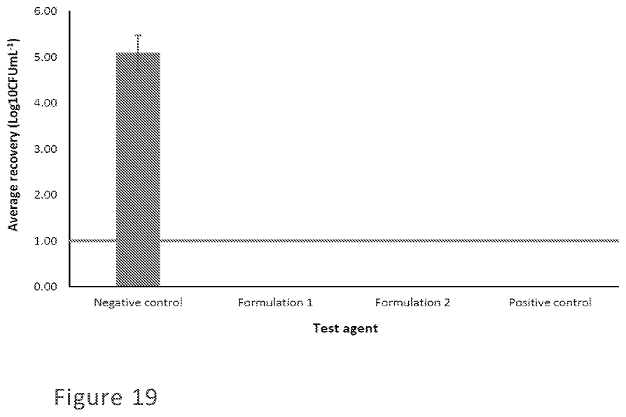

For the Staphylococcus aureus 72 hour pre-formed biofilms an average of 5.10±0.38 Log₁₀CFUsample⁻¹ viable S. aureus were recovered from the negative control (Table 36, FIG. 19). No viable Staphylococcus aureus was recovered following treatment for 24 hours with Formulation 1 or Formulation 2.

TABLE 36
Average quantities of viable Staphylococcus aureus
recovered and average reductions following treatment for 24 hours
Formulation 1-0.2% copper gluconate and 0.1% zinc gluconate
with Lactobacillus plantarum and Formulation 2-0.2%
copper gluconate and 0.1% zinc gluconate, when compared to
the negative control.

| | Average recovery ± SD | Average reduction ± SD |
|---|---|---|
| Test agent | (Log₁₀CFUsample⁻¹) | (Log₁₀CFUsample⁻¹) |
| Negative control | 5.10 ± 0.38 | N/A |
| Formulation 1 | 0.00 ± 0.00 | 5.10 ± 0.00* |
| Formulation 2 | 0.00 ± 0.00 | 5.10 ± 0.00* |
| Positive control | 0.00 ± 0.00 | 5.10 ± 0.00* |

SD = standard deviation,
N/A = not applicable,
*= p < 0.001.

Pseudomonas aeruginosa

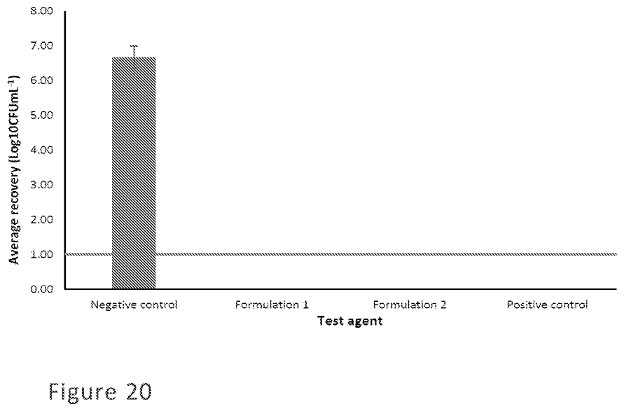

For the Pseudomonas aeruginosa 72 hour pre-formed biofilms, an average of 6.68±0.32 Log₁₀CFUsample⁻¹ viable P. aeruginosa were recovered from the negative control (Table 37, FIG. 20). No viable P. aeruginosa was recovered following treatment for 24 hours with Formulation 1 or Formulation 2.

TABLE 37
Average quantities of viable Pseudomonas aeruginosa
recovered and average reductions following treatment for 24 hours
with Formulation 1-0.2% copper gluconate and 0.1% zinc gluconate
with Lactobacillus plantarum and Formulation 2-0.2%
copper gluconate and 0.1% zinc gluconate, when compared to
the negative control.

| | Average recovery ± SD | Average reduction ± SD |
|---|---|---|
| Test agent | (Log₁₀CFUsample⁻¹) | (Log₁₀CFUsample⁻¹) |
| Negative control | 6.68 ± 0.32 | N/A |
| Formulation 1 | 0.00 ± 0.00 | 6.68 ± 0.00* |
| Formulation 2 | 0.00 ± 0.00 | 6.68 ± 0.00* |
| Positive control | 0.00 ± 0.00 | 6.68 ± 0.00* |

SD = standard deviation,
N/A = not applicable,
*= p < 0.001.

Candida albicans

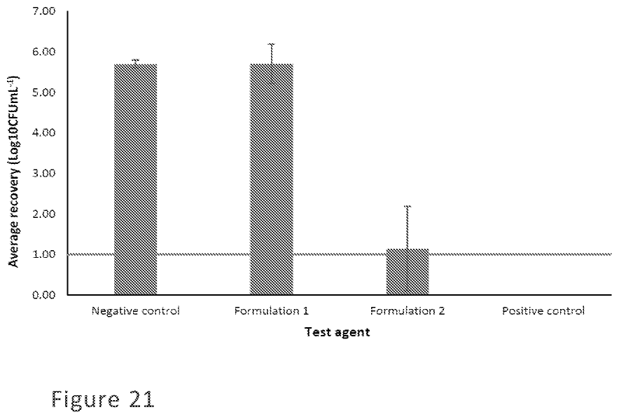

For the Candida albicans 72 hour pre-formed biofilms, an average of 5.70±0.10 Log₁₀CFUsample⁻¹ viable C. albicans were recovered from the negative control (Table 38, FIG. 21). Following treatment for 24 hours, Formulation 2 achieved an average Log reduction of 4.56±1.04 Log₁₀CFUsample⁻¹, when compared to the negative control. No reduction was observed following treatment with Formulation 1.

TABLE 38
Average quantities of viable Candida albicans
recovered and average reductions following treatment for 72 hours
with Formulation 1-0.2% copper gluconate and 0.1% zinc gluconate
with Lactobacillus plantarum and Formulation 2-0.2%
copper gluconate and 0.1% zinc gluconate, when compared to
the negative control.

| | Average recovery ± SD | Average reduction ± SD |
|---|---|---|
| Test agent | (Log₁₀CFUsample⁻¹) | (Log₁₀CFUsample⁻¹) |
| Negative control | 5.70 ± 0.10 | N/A |
| Formulation 1 | 5.71 ± 0.48 | No reduction |
| Formulation 2 | 1.14 ± 1.04 | 4.56 ± 1.04* |
| Positive control | 0.00 ± 0.00 | 5.70 ± 0.00** |

SD = standard deviation,
N/A = not applicable,
*= p < 0.01,
**= p < 0.001.

### Discussion

No viable S. aureus or P. Aeruginosa were recovered following treatment with Formulation 1 and Formulation 2 for 24 hour and 72 hour pre-formed biofilms. Treatment with Formulation 2 resulted in a >4 Log reduction of C. albicans. Treatment with Formulation 1 did not result in a Log reduction of C. albicans suggesting the presence of L. plantarum may inhibit the antibiofilm effects of zinc gluconate and copper gluconate against C. albicans.

1. A composition comprising (a) a zinc salt and (b) a copper salt, wherein the zinc salt is zinc gluconate and the copper salt is copper gluconate.

2. A composition according to claim 1, which comprises zinc gluconate in a concentration:
(ii) of at least 0.05% w/v e.g. 0.055, 0.06, 0.065, 0.07, 0.075, 0.08, 0.085, 0.09, 0.095%;
(ii) approx. 0.1% w/v; or
(iii) greater than 0.1% w/v, e.g. 0.125, 0.13, 0.014, 0.015, 0.016, 0.017, 0.018, 0.019%, 0.02% w/v or greater.

3. (canceled)

4. (canceled)

5. A composition according to claim 1, which comprises copper gluconate in a concentration of:
(i) at least 0.1% w/v e.g. 0.125, 0.13, 0.014, 0.015, 0.016, 0.017, 0.018, 0.019% w/v or greater;
(ii) about 0.2% w/v; or
(iii) greater than 0.2% e.g. 0.21, 0.22, 0.23, 0.24, 0.25, 0.26, 0.27, 0.28, 0.29 or 0.30% w/v or greater.

6. (canceled)

7. (canceled)

8. A composition according to claim 1, which further comprises one or more further components comprising:
(i) one or more further copper components, for example, copper sulfate, a copper-complex e.g. a copper chelates e.g. copper histidine;
(ii) one or more further zinc components e.g. zinc sulfate and/or one or more zinc complexes e.g. zinc histidine;
(iii) one or more titanium compounds e.g. titanium dioxide; or
(iv) one or more further pro-biotic bacteria and/or yeast.

9.-12. (canceled)

13. A composition according to claim 8, wherein:
(i) the probiotic bacteria is selected from Lactobacillus species, bifidobacterium species, e.g. Lactobacillus rhamnosus, Lactobacillus casei, Lactobacillus acidophilus and Lactobacillus lactus; or
iii) the probiotic yeast is Saccharomyces boulardii.

14. (canceled)

15. A composition according to claim 1:
(i) which is a suspension;
(ii) which further comprises Lactobacillus plantarum;
(iii) which comprises Lactobacillus plantarum at a concentration of about 1×10⁸±5×10⁷ CFUmL−1;
(iv) wherein the Lactobacillus plantarum is a sterilized Lactobacillus plantarum; or
(v) which is for use as an anti-microbial agent.

16.-19. (canceled)

20. A composition according to claim 15, which is for use in the prevention and/or treatment of an infection in a subject of one or more of the following micro-organisms:
a) Acinetobacter baumannii;
b) Campylobacter coli;
c) Haemophilus influenzae;
d) Helicobacter aeruginosa;
e) Pseudomonas aeruginosa;
f) Salmonella enteritidis;
g) Enterococcus faecium;
h) Neisseria gonorrhoeae;
i) Stomatococcus mucilaginous;
j) Staphylococcus aureus;
k) Streptococcus pneumoniae;
l) Staphylococcus hominis;
m) Streptococcus mutans;
n) Cutibacterium;
o) Anaerococcus;
p) Peptoniphilus;
q) Candida albicans;
r) Malassezia restricta;
s) Malassezia globosa;
t) Propionibacterium;
u) Gardnerella vaginalis;
v) Escherichia coli;
w) Prevotella;
x) Peptostreptococcus;
y) Bacteroides; and
z) Corynebacterium.

21. A composition according to claim 20, which is for use in the prevention and/or treatment of:
(i) an Acinetobacter baumannii infection in a subject;
(ii) a Campylobacter coli infection in a subject;
(iii) a Haemophilus influenzae infection in a subject;
(iv) a Helicobacter aeruginosa infection in a subject;
(v) a Pseudomonas aeruginosa infection in a subject;
(vi) a Salmonella enteritidis infection in a subject;
(vii) a Enterococcus faecium infection in a subject;
(viii) a Neisseria gonorrhoeae infection in a subject;
(ix) a Staphylococcus aureus infection in a subject; or
(x) a Streptococcus pneumoniae infection in a subject.

22.-30. (canceled)

31. A composition according to any of claims 1 to 19 claim 1, which is for use in the prevention and/or treatment of a disorder characterised by colonization by:
(i) C. albicans, C. auris and/or A. brasiliensis; or
(ii) S. aureus and P. aeruginosa.

32. (canceled)

33. A composition according to claim 1, which is for use to prevent and/or treat a disorder characterized by colonization by one or more of the following micro-organisms:
a) Acinetobacter baumannii;
b) Campylobacter coli;
c) Haemophilus influenzae;
d) Helicobacter aeruginosa;
e) Pseudomonas aeruginosa;
f) Salmonella enteritidis;
g) Enterococcus faecium;
h) Neisseria gonorrhoeae;
i) Stomatococcus mucilaginous;
j) Staphylococcus aureus;
k) Streptococcus pneumoniae;
l) Staphylococcus hominis;
m) Streptococcus mutans;
n) Cutibacterium;
o) Anaerococcus;
p) Peptoniphilus;
q) Candida albicans;
r) Malassezia restricta;
s) Malassezia globosa;
t) Propionibacterium;
u) Gardnerella vaginalis;
v) Escherichia coli;
w) Prevotella;
x) Peptostreptococcus;
y) Bacteroides; and
z) Corynebacterium: and
ai) Methicillin-resistant Staphylococcus aureus.

34. A composition according to claim 33, wherein the wound is a skin wound selected from the group consisting of a laceration, a penetrating wound, a venous stasis ulcer, a pressure ulcer, a surgical wound and an open full-thickness skin puncture wound.

35. A composition according to claim 1 for use in the prevention and/or treatment of:
(i) a fungal infection in a subject, wherein optionally the fungal infection is a Candida infection and/or the composition is for use to treat an oral Candida albicans and/or a Candida auris infection, and/or a vaginal Candida albicans and/or a Candida auris infection;
(ii) a genital tract disorder and/or genital tract infection.

36.-38. (canceled)

39. A composition according to claim 1, which is:
(i) for topical, oral and/or parenteral administration;
(ii) for topical administration; or
(iii) for use to prevent or reduce the formation of a biofilm.

40. (canceled)

41. (canceled)

42. A combination comprising (a) zinc salt and (b) copper Galt, salt:
(i) wherein the zinc salt is zinc gluconate and the copper salt is copper gluconate;
(ii) which further comprises one or more of the features as recited in claim 8.

43. (canceled)

44. A method of treating and/or preventing a microbial infection in a subject, the method comprising:
a) administering a composition according to claim 1 to a subject in need thereof.

45. A method according to claim 44, wherein the microbial infection is selected from the group consisting of:
a) Acinetobacter baumannii;
b) Campylobacter coli;
c) Haemophilus influenzae;
d) Helicobacter aeruginosa;
e) Pseudomonas aeruginosa;
f) Salmonella enteritidis;
g) Enterococcus faecium;
h) Neisseria gonorrhoeae;
i) Stomatococcus mucilaginous;
j) Staphylococcus aureus;
k) Streptococcus pneumoniae;
l) Staphylococcus hominis;
m) Streptococcus mutans;
n) Cutibacterium;
o) Anaerococcus;
p) Peptoniphilus;
q) Candida albicans;
r) Malassezia restricta;
s) Malassezia globosa;
t) Propionibacterium;
u) Gardnerella vaginalis;
v) Escherichia coli;
w) Prevotella;
x) Peptostreptococcus.
y) Bacteroides;
z) Corynebacterium; and
ai) Methicillin-resistant Staphylococcus aureus.

46. A method of treating:
(i) a wound, lesion and/or burn suspected of being infected by a micro-organism and/or preventing infection of a wound, lesion and/or burn of a subject by a micro-organism; or
(ii) a wound which is a skin wound selected from the group consisting of a laceration, a penetrating wound, a venous stasis ulcer, a pressure ulcer, a surgical wound and an open full-thickness skin puncture wound;
the method comprising:
a) administering a composition according to claim 1 to a site of a wound, a lesion and/or a burn of a subject in need thereof.

47. (canceled)

48. A method of treating and/or preventing a genital tract infection, the method comprising administering a composition according to claim 1 to a site in the genital tract.

49. A composition according to claim 1 which is for use in the treatment and/or prevention of one or more of the following:
(a) body odour;
(b) dandruff;
(c) dental caries;
(d) genital infections; and
(e) halitosis.

50. A composition according to claim 1, wherein the composition is a chewable product.
